(19) United States
(12) Patent Application Publication
(10) Pub. No.: US 2022/0002283 A1
(43) Pub. Date: Jan. 6, 2022

(54) MELOXICAM CO-CRYSTALS

(71) Applicant: Mylan Laboratories Limited, Hyderabad (IN)

(72) Inventors: Ramakoteswara Rao JETTI, Hyderabad (IN); Srinivas ENUGULA, Hyderabad (IN); Subramanyam DANDALA, Hyderabad (IN); Sureshbabu JAYACHANDRA, Hyderabad (IN); Vijaya Krishna RAVI, Hyderabad (IN); Venkata Naga Vikas Chandra Dev RAVI, Hyderabad (IN)

(73) Assignee: Mylan Laboratories Limited, Hyderabad (IN)

(21) Appl. No.: 17/291,101

(22) PCT Filed: Nov. 4, 2019
(86) PCT No.: PCT/IN2019/050815

§ 371 Date: May 4, 2021

(51) Int. Cl. C07D 417/12

(52) U.S. Cl. CPC C07D 417/12; C07B 2200/13

(57) ABSTRACT

Co-crystals of meloxicam co-formers can be prepared by co-crystallization from a polar solvent, such as aqueous dimethyl sulfoxide; or by slurry processes, such as with ethyl acetate. Such co-crystals have improved purities and are physically stable under storage for several months.

### CROSS-REFERENCE TO RELATED APPLICATIONS

This application claims priority to the filing dates of Indian Provisional Application No. 201841041849, filed on Nov. 5, 2018; Indian Provisional Application No. 201941017796, filed on May 3, 2019; and Indian Provisional Application No. 201941017804, filed on May 3, 2019; each of the preceding are hereby incorporated by reference in their entireties.

### BACKGROUND OF THE INVENTION

### Field of the Disclosure

The present disclosure relates to co-crystals of meloxicam having improved purities and processes for preparing the same.

### Description of the Related Art

Meloxicam is known as 4-hydroxy-2-methyl-N-(5-methyl-2-thiazolyl)-2H-1,2-benzothiazine-3-carboxamide-1,1-dioxide. Meloxicam is depicted by the following chemical structure of formula 1:

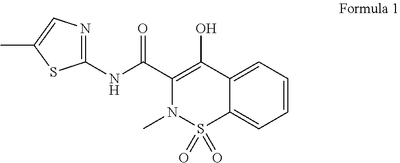 [SMILES: Cc1cnc(NC(=O)C2=C(O)c3ccccc3S(=O)(=O)N2C)s1]

Meloxicam is a non-steroidal, anti-inflammatory (NSAID) and anti-pyretic drug that is presently used to relieve symptoms of arthritis, fever, and can be used as an analgesic for inflammatory conditions. It was developed originally by Boehringer Ingelheim and marketed in Europe under brand names such as Melox, Movalis, and Recoxa for the treatment of rheumatoid arthritis, short term use for osteoarthritis, and for ankylosing spondylitis. In the United States, it is marketed as Mobic® and Vivlodex® for the treatment of osteoarthritis and Qmiiz™ ODT for the treatment of osteoarthritis in adults and rheumatoid arthritis for all ages. Meloxicam is commercialized as a tablet, oral disintegrating tablet, and capsule, each at 7.5 and 15 mg per dose, and as an oral suspension at 7.5 mg/5 mL per dose.

U.S. Pat. Nos. 8,124,603 and 8,389,512 disclose various co-crystals of meloxicam, including those with fumaric acid, succinic acid, adipic acid, benzoic acid, DL-malic acid, L-malic acid, glutaric acid, acetylsalicylic acid, salicylic acid, 1-hydroxy-2-naphthoic acid, maleic acid, 4-hydroxybenzoic acid, malonic acid, glycolic acid, 2,5-dihydroxybenzoic acid, camphoric acid, maltol, ethyl maltol, and hydrocinnamic acid.

### SUMMARY OF THE DISCLOSURE

The present disclosure provides processes and co-crystal products having reduced residual impurities, including excess, uncomplexed/residual meloxicam or co-former (i.e., “free API” and/or “free co-former” as defined herein). Through extensive research, processes for substantially eliminating such impurities have been developed which yield meloxicam co-crystals that are substantially pure with respect to free API, free co-former, and/or residual solvents. Such processes are suitable for large scale co-crystal production.

In one aspect, the present disclosure provides a “substantially pure” meloxicam co-crystal where “substantially pure” is defined herein with respect to free API, free co-former, and/or residual solvent. For example, a meloxicam co-crystal can be “substantially free of free meloxicam” when a PXRD spectrum of the co-crystal is absent of one or more signals attributable to free meloxicam selected from the group consisting of 6.5, 11.2, 13.2, 14.9, and 17.8°+/−0.2° 2Θ when collected according to the process described below. Absence of any one of the characteristic 2Θ peaks of meloxicam substantiate the absence of free meloxicam in the co-crystals and further confirms the phase purity of co-crystal.

In another aspect, the present disclosure provides a process for preparing meloxicam co-crystals suitable for commercial scale production comprising forming a solution of meloxicam and a co-former in an organic solvent, combining the solution with an anti-solvent, and isolating the meloxicam co-crystal.

In another aspect, the present disclosure provides a process for preparing meloxicam co-crystals suitable for commercial scale production comprising forming a suspension of meloxicam and a co-former in an organic solvent, agitating the suspension (e.g., stirring) for a period of time suitable to provide a meloxicam co-crystal, and isolating the meloxicam co-crystal, wherein either the ratio of meloxicam measured in grams to organic solvent measured in mL (“the w/v ratio”) in the suspension is greater than about 1:5; or one of the meloxicam or the co-former is present in the suspension in at least a 10% molar excess with respect to the other.

### BRIEF DESCRIPTION OF THE DRAWINGS

FIG. 1 is a powder X-ray diffractogram (PXRD) of a meloxicam and acetylsalicylic acid (1:1) co-crystal prepared according to Example 1.

FIG. 2 is a powder X-ray diffractogram (PXRD) of a meloxicam and 1-hydroxy-2-naphthoic acid (1:1) co-crystal prepared according to Example 2.

FIG. 3 is a powder X-ray diffractogram (PXRD) of a meloxicam and salicylic acid Form III (1:1) co-crystal prepared according to Example 3.

FIG. 4 is a comparison of the powder X-ray diffractograms (PXRDs) of (a) meloxicam (form I); (b) the meloxicam:acetylsalicylic acid co-crystal (1:1) prepared according to Example 1; and (c) the meloxicam:acetylsalicylic acid co-crystal (1:1) prepared according to the prior art process disclosed in Example 43 of U.S. Pat. No. 8,124,603; the arrows indicate characteristic peaks for free meloxicam impurities within the product of the prior art process.

FIG. 5 is a comparison of the powder X-ray diffractograms (PXRDs) of (a) meloxicam (form I); (b) the meloxicam:salicylic acid Form III co-crystal (1:1) prepared according to Example 3; and (c) the meloxicam:salicylic acid Form III co-crystal (1:1) prepared according to the prior art process disclosed in Example 44 of U.S. Pat. No. 8,124,603; the arrows indicate characteristic peaks for free meloxicam impurities within the product of the prior art process.

FIG. 6 is a comparison of the powder X-ray diffractograms (PXRDs) of (a) meloxicam (form I); (b) the meloxicam:1-hydroxy-2-naphthoic acid co-crystal (1:1) prepared according to Example 2; and (c) the meloxicam:1-hydroxy-2-naphthoic acid co-crystal (1:1) prepared according to the prior art process disclosed in Example 45 of U.S. Pat. No. 8,124,603; the arrows indicate characteristic peaks for free meloxicam impurities within the product of the prior art process.

FIG. 7 is a powder X-ray diffractogram (PXRD) of a meloxicam:1-hydroxy-2-naphthoic acid (1:1) co-crystal obtained as per Example 6.

FIG. 8 is a powder X-ray diffractogram (PXRD) of a meloxicam:1-hydroxy-2-naphthoic acid (1:1) co-crystal obtained as per Example 7.

FIG. 9 is a comparison of the powder X-ray diffractograms (PXRDs) of (a) meloxicam (form I); (b) the meloxicam:acetylsalicylic acid co-crystal (1:1) prepared according to Example 8; and (c) the meloxicam:acetylsalicylic acid co-crystal (1:1) prepared according to the prior art process disclosed in Example 43 of U.S. Pat. No. 8,124,603; the arrows indicate characteristic peaks for free meloxicam impurities within the product of the prior art process.

FIG. 10 is a comparison of the powder X-ray diffractograms (PXRDs) of (a) meloxicam (form I); (b) the meloxicam:salicylic acid Form III co-crystal (1:1) prepared according to Example 9; and (c) the meloxicam:salicylic acid Form III co-crystal (1:1) prepared according to the prior art process disclosed in Example 44 of U.S. Pat. No. 8,124,603; the arrows indicate characteristic peaks for free meloxicam impurities within the product of the prior art process.

FIG. 11 is a comparison of the powder X-ray diffractograms (PXRDs) of (a) meloxicam (form I); (b) the meloxicam:maleic acid co-crystal (1:1) prepared according to Example 10; and (c) the meloxicam:maleic acid co-crystal (1:1) prepared according to the prior art process disclosed in Example 42 of U.S. Pat. No. 8,124,603; the arrows indicate characteristic peaks for free meloxicam impurities within the product of the prior art process.

FIG. 12 is a comparison of the powder X-ray diffractograms (PXRDs) of (a) meloxicam (form I); (b) the meloxicam:succinic co-crystal (2:1) prepared according to Example 11; and (c) the meloxicam:succinic acid co-crystal (2:1) prepared according to the prior art process disclosed in Example 46 of U.S. Pat. No. 8,124,603.

FIG. 13a is a DSC thermogram of meloxicam:succinic co-crystal (2:1) prepared according to Example 12 before water leaching; note the presence of trace amounts of succinic acid contamination (endotherm at about 187° C.).

FIG. 13b is a DSC thermogram of meloxicam:succinic co-crystal (2:1) prepared according to Example 12 after water leaching at 50±5° C.; note the completely absence of amounts of succinic acid contamination (lack of endotherm at about 187° C.).

FIG. 14 is a powder X-ray diffractogram (PXRD) of a meloxicam and acetylsalicylic acid (1:1) co-crystal obtained as per Example 13.

FIG. 15 is a powder X-ray diffractogram (PXRD) of a meloxicam and salicylic acid Form III (1:1) co-crystal obtained as per Example 14.

FIG. 16 is a powder X-ray diffractogram (PXRD) of a meloxicam and maleic acid (1:1) co-crystal obtained as per Example 15.

FIG. 17 is a powder X-ray diffractogram (PXRD) of a meloxicam and succinic acid (2:1) co-crystal obtained as per Example 16.

FIG. 18 is a powder X-ray diffractogram (PXRD) of a meloxicam and succinic acid (2:1) co-crystal obtained as per Example 17.

### DETAILED DESCRIPTION OF THE DISCLOSURE

It has been noted that meloxicam co-crystals prepared according to the processes disclosed in, for example, U.S. Pat. Nos. 8,124,603 and 8,389,512, can result in co-crystal products having significant residual impurities, including excess, uncomplexed/residual meloxicam or co-former (i.e., “free API” and/or “free co-former” as defined herein). Through extensive research, processes for substantially eliminating such excess impurities has been developed which yields, for the first time, meloxicam co-crystals that are substantially pure with respect to free API, free co-former, and/or residual solvents.

“Co-crystal” as used herein means a crystalline material composed of two or more different molecules that co-exist in the crystalline unit cell with a defined stoichiometry and interact non-ionically and non-covalently. Herein, the co-crystals comprise at least one active pharmaceutical ingredient (API) and at least one co-crystal former (“co-former”). In certain embodiments, the “co-crystal” herein is a crystalline material composed of one active pharmaceutical ingredient (API) and one co-crystal former (“co-former”). “Non-ionic” as used herein refers to energetically favorable molecular interactions that are not considered an ionic bond or a fully covalent bond, and includes, for example, hydrogen bonding.

The co-former may be selected from, for example, carboxylic acids having a pKa between about 2.95 and 4.70, or between about 2.95 and 3.55, such as the group consisting of fumaric acid, succinic acid, adipic acid, benzoic acid, DL-malic acid, L-malic acid, glutaric acid, acetylsalicylic acid, salicylic acid, 1-hydroxy-2-naphthoic acid, maleic acid, 4-hydroxybenzoic acid, malonic acid, glycolic acid, 2,5-dihydroxybenzoic acid, camphoric acid, maltol, ethyl maltol, and hydrocinnamic acid.

In one embodiment, the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, and salicylic acid. In another embodiment, the co-former is selected from the group consisting of fumaric acid, succinic acid, adipic acid, DL-malic acid, L-malic acid, glutaric acid, maleic acid, malonic acid, glycolic acid, camphoric acid, maltol, and ethyl maltol.

In one embodiment, the co-former is 1-hydroxy-2-naphthoic acid, acetylsalicylic acid or salicylic acid, succinic acid, or maleic acid. In another embodiment, the co-former is 1-hydroxy-2-naphthoic acid. In another embodiment, the co-former is acetylsalicylic acid. In another embodiment, the co-former is salicylic acid. In another embodiment, the co-former is succinic acid. In another embodiment, the co-former is maleic acid.

Each of the preceding co-formers can form co-crystals with meloxicam. One way of determining whether a composition is a co-crystal or a salt is to consider the difference in pKa values for the constituents. For example, where an API and its co-former have a ΔpKa (i.e., pKa (conjugate acid of base)−pKa (acid)) greater than or equal to 3, substantial proton transfer results in ionization and salt formation. However, when both API and co-former have ionizable functional groups, and the API and its co-former have a ΔpKa (i.e., pKa (conjugate acid of base)−pKa (acid)) less than or equal to about 1.5 (e.g., about 1.2 or about 1.0), there can be less than substantial proton transfer and the interaction may be considered non-ionic.

| | pKa | Delta pKa |
|---|---|---|
| Co-Former | (co-former) | (base* − acid) |
| Gentisic acid (2,5-Dihydroxybenzoic acid) | 2.93 | 1.27 |
| 4-Hydroxybenzoic acid | 4.58 | −0.38 |
| Acetylsalicylic acid | 3.50 | 0.7 |
| 1-Hydroxy-2-naphthoic acid | 2.70 | 1.5 |
| Salicylic acid | 2.97 | 1.23 |
| Benzoic acid | 4.204 | −0.004 |
| Succinic acid | 4.19 | −0.01 |
| Maleic acid | 1.93 | 2.25 |
| Hydrocinnamic acid | 4.66 | −0.46 |

*pKa value of meloxicam is 4.2 (—OH)

In general, the “co-former” is not a solvent. Examples of solvents that are not co-formers include, but are not limited to, water, methanol, ethanol, isopropanol, propanol, butanol, dimethyl sulfoxide, ethyl acetate, isopropyl acetate, acetone, butanone, dimethylformamide, dimethylacetamide, N-methylpyrrolidinone, tetrahydrofuran, dioxane, chloroform, dichloromethane, propylene glycol, ethylene glycol, dimethyl carbonate, diethyl carbonate, ethylene carbonate, propylene carbonate, toluene, and xylene(s).

“Substantially pure” as used herein means the substance (e.g., co-crystal) contains less than about 1 wt. % of any one impurity, and/or less than about 2.5 wt. % of total impurities, and/or less than 5000 ppm of any one residual solvent.

“About” as used herein means +/−10% of the referenced value. In certain embodiments, “about” means +/−9%, or +/−8%, or +/−7%, or +/−6%, or +/−5%, or +/−4%, or +/−3%, or +/−2+/− or +/−1% of the referenced value.

In certain embodiments, “substantially pure” means the substance (e.g., co-crystal) contains less than about 2 wt. %, or less than about 1 wt. %, or less than about 0.5 wt. % of total impurities, or less than about 0.3 wt. % of total impurities.

In certain embodiments, “substantially pure” means the substance (e.g., co-crystal) contains less than about 1.0 wt. %, or less than 0.5 wt. %, or less than about 0.3 wt. %, or less than about 0.15 wt. %, or less than about 0.10 wt. %, or less than about 0.05 wt. % of any one impurity.

In yet other embodiments, “substantially pure” means the substance (e.g., co-crystal) contains less than about 1.0 wt. %, or less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

“Free API” and “free co-former” refer to API and co-former molecules, respectively, that are not complexed with one another within the crystalline unit cell of the co-crystal. Free API and/or free co-former may be identified within the co-crystal, for example, by identification through powder X-ray diffraction spectroscopy (PXRD), thermogravimetric analysis (TGA), and/or differential scanning calorimetry (DSC) analyses, as would be familiar to those skilled in the art. In one embodiment, the free API and/or free co-former may be identified by powder X-ray diffraction spectroscopy (PXRD) as described in the section entitled “X-ray powder diffraction analysis of meloxicam co-crystals”, infra.

In yet other embodiments, “substantially pure” means the substance (e.g., co-crystal) contains less than about 1.0 wt. %, or less than about 0.5 wt. %, or less than about 0.3 wt. %, or less than about 0.15 wt. %, or less than about 0.10 wt. %, or less than about 0.05 wt. % of “free API”, e.g., meloxicam.

In yet other embodiments, “substantially pure” means the substance (e.g., co-crystal) contains less than about 1.0 wt. %, or less than about 0.5 wt. %, or less than about 0.3 wt. %, or less than about 0.15 wt. %, or less than about 0.10 wt. %, or less than about 0.05 wt. % of any one impurity that is “free co-former.”

In yet other embodiments, a “substantially pure” meloxicam co-crystal is characterized by an absence of PXRD signals attributable to free meloxicam. For example, the PXRD for the meloxicam co-crystal can be characterized by an absence of one or more PXRD signals attributable to free meloxicam selected from the group consisting of 6.5, 11.2, 13.2, 14.9, and 17.8°+/−0.2 2Θ as measured according to the “X-ray powder diffraction analysis of meloxicam co-crystals (PXRD)” section below.

For example,

a “substantially pure” meloxicam:1-Hydroxy-2-Naphthoic acid co-crystal (1:1) can be characterized by an absence of a PXRD signal attributable to free meloxicam at 13.2°+/−0.2 2 theta (“2Θ”);

a “substantially pure” meloxicam:acetylsalicylic acid co-crystal (1:1) can be characterized by an absence of a PXRD signal attributable to free meloxicam at 11.2°+/−0.2 2Θ;

a “substantially pure” meloxicam:salicylic acid co-crystal (1:1) can be characterized by an absence of a PXRD signal attributable to free meloxicam at 6.5°+/−0.2 2Θ;

a “substantially pure” meloxicam:succinic acid crystal (2:1) can be characterized by an absence of a PXRD signal attributable to free meloxicam at 13.2°+/−0.2 2Θ; and

a “substantially pure” meloxicam:maleic acid crystal (1:1) can be characterized by an absence of a PXRD signal attributable to free meloxicam at 6.5°+/−0.2 2Θ.

In yet other embodiments, “substantially pure” means the substance (e.g., co-crystal) contains less than about 5000 ppm, or less than about 4500 ppm, or less than about 4000 ppm, or less than about 3500 ppm, or less than about 3000 ppm, or less than about 2000 ppm, or less than about 1000 ppm, or less than about 500 ppm of any one residual solvent. Such residual solvents may be any one of the organic solvents and/or anti-solvents described in the following processes.

In certain embodiments, the residual solvent is dimethyl sulfoxide, and the co-crystal contains between about 100 ppm and about 5000 ppm dimethyl sulfoxide; or between about 100 ppm and about 4000 ppm dimethyl sulfoxide; or between about 100 ppm and about 3000 ppm dimethyl sulfoxide; or between about 100 ppm and about 2000 ppm dimethyl sulfoxide; or between about 100 ppm and about 1000 ppm dimethyl sulfoxide; or between about 300 ppm and about 5000 ppm dimethyl sulfoxide; or between about 300 ppm and about 4000 ppm dimethyl sulfoxide; or between about 300 ppm and about 3000 ppm dimethyl sulfoxide; or between about 300 ppm and about 2000 ppm dimethyl sulfoxide; or between about 300 ppm and about 1000 ppm dimethyl sulfoxide.

In certain embodiments, the residual solvent is dimethyl sulfoxide, the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is dimethyl sulfoxide, the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is dimethyl sulfoxide, the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is dimethyl sulfoxide, the co-former is salicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is dimethyl sulfoxide, the co-former is salicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is dimethyl sulfoxide, the co-former is salicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is dimethyl sulfoxide, the co-former is acetylsalicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is dimethyl sulfoxide, the co-former is acetylsalicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is dimethyl sulfoxide, the co-former is acetylsalicylic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is dimethyl sulfoxide, the co-former is 1-hydroxy-2-naphthoic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is dimethyl sulfoxide, the co-former is 1-hydroxy-2-naphthoic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is dimethyl sulfoxide, the co-former is 1-hydroxy-2-naphthoic acid, and the co-crystal contains (a) between about 100 ppm and about 5000 ppm dimethyl sulfoxide; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In other embodiments, the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In other embodiments, the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-former is salicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In another embodiment, the co-former is salicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In another embodiment, the co-former is salicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-former is acetylsalicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In another embodiment, the co-former is acetylsalicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In another embodiment, the co-former is acetylsalicylic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-former is 1-hydroxy-2-naphthoic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In another embodiment, the co-former is 1-hydroxy-2-naphthoic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In another embodiment, the co-former is 1-hydroxy-2-naphthoic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-former is succinic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In another embodiment, the co-former is succinic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In another embodiment, the co-former is succinic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain embodiments, the co-former is maleic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former. In another embodiment, the co-former is maleic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free meloxicam. In another embodiment, the co-former is maleic acid, and the co-crystal contains less than about 0.5 wt. %, or less than about 0.3 wt. %, or less than about 0.15 wt. %, or less than about 0.10 wt. %, or less than about 0.05 wt. % of any one impurity that is free co-former.

In certain other embodiments, the residual solvent is ethyl acetate, and the co-crystal contains between about 100 ppm and about 5000 ppm ethyl acetate; or between about 100 ppm and about 4000 ppm ethyl acetate; or between about 100 ppm and about 3000 ppm ethyl acetate; or between about 100 ppm and about 2000 ppm ethyl acetate; or between about 100 ppm and about 1000 ppm ethyl acetate; or between about 300 ppm and about 5000 ppm ethyl acetate; or between about 300 ppm and about 4000 ppm ethyl acetate; or between about 300 ppm and about 3000 ppm ethyl acetate; or between about 300 ppm and about 2000 ppm ethyl acetate; or between about 300 ppm and about 1000 ppm ethyl acetate.

In certain embodiments, the residual solvent is ethyl acetate, the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is ethyl acetate, the co-former is salicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-former is salicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-former is salicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is ethyl acetate, the co-former is acetylsalicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-former is acetylsalicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-former is acetylsalicylic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is ethyl acetate, the co-former is 1-hydroxy-2-naphthoic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-former is 1-hydroxy-2-naphthoic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-former is 1-hydroxy-2-naphthoic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is ethyl acetate, the co-former is succinic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-former is succinic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-former is succinic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

In certain embodiments, the residual solvent is ethyl acetate, the co-former is maleic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free API (e.g., meloxicam) and/or free co-former.

In certain embodiments, the residual solvent is ethyl acetate, the co-former is maleic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free meloxicam.

In certain embodiments, the residual solvent is ethyl acetate, the co-former is maleic acid, and the co-crystal contains (a) between about 100 ppm and about 5000 ppm ethyl acetate; and (b) less than about 1.0 wt. %, or about 0.5 wt. %, or less than about 0.3 wt. %, or less than about 0.15 wt. %, or less than about 0.10 wt. %, or less than about 0.05 wt. % of any one impurity that is free co-former.

Notably, the substantially pure meloxicam co-crystals provided herein are storage stable. “Storage stable” as used herein means that a sample of the co-crystal, following storage under the recited conditions, has less than 1 wt. % (e.g., less than 0.5 wt. % or less than 0.2 wt. % or less than 0.15 wt. %) total impurities as measured by HPLC. In certain embodiments, a storage stable co-crystal has between 0.05 wt. % and 1 wt. % or between 0.05 wt. % and 0.5 wt. % or between 0.05 wt. % and 0.2 wt. % or between 0.05 wt. % and 0.15 wt. % total impurities as measured by HPLC.

A storage stable co-crystal, in additional embodiments, following storage under the recited conditions, may have less than 0.1 wt. % (e.g., less than 0.05 wt. % or less than 0.03 wt. %) of any one individual impurity as measured by HPLC; for example, between about 0.03 wt. % and 0.1 wt. % of any one individual impurity as measured by HPLC. Examples of individual impurity include methyl meloxicam, ethyl meloxicam, and Meloxicam Related Compounds A, B, C, and D (per the Meloxicam USP Monograph, USP35-3789). USP Meloxicam Related Compound A is ethyl 4-hydroxy-2-methyl-2H-1,2-benzothiazine-3-carboxylate 1,1-dioxide; USP Meloxicam Related Compound B is 5-Methylthiazol-2-amine; USP Meloxicam Related Compound C is Isopropyl-4-hydroxy-2-methyl-2H-1,2-benzothiazine-3-carboxylate-1,1-dioxide; and USP Meloxicam Related Compound D is 4-methoxy-2-methyl-N-(5-methyl-1,3-thiazole-2-yl)-2H-1,2-benzothiazine-3-carboxamide-1,1-dioxide.

In one example, the substantially pure meloxicam co-crystals provided herein are storage stable upon storage at 25° C.±2° C./60%±5% relative humidity (RH) for 3, 6, 9, and/or 12 months. In another example, the substantially pure meloxicam co-crystals provided herein are storage stable upon storage at 40° C.±2° C./75%±5% relative humidity (“RH”) for 1, 2, 3, and/or 6 months.

In another example, the substantially pure meloxicam co-crystals provided herein are storage stable upon storage at 25° C.±2° C./60%±5% relative humidity (“RH”) and/or 40° C.±2° C./75%±5% relative humidity (“RH”) such that Meloxicam Related Compounds A, B, C, and D, methyl meloxicam, and ethyl meloxicam are each present following storage at less than 0.1 wt. %, or less than 0.05 wt. % or less than 0.03 wt. %, or are not detectable, each by HPLC. For example, Meloxicam Related Compounds, A, B, C, and D may be essentially undetectable following storage and methyl meloxicam and ethyl meloxicam are each present following storage at less than 0.1 wt. %, or less than 0.05 wt. % or less than 0.03 wt. %, each by HPLC.

### Co-Crystallization by Crystallization Process (Process 1)

Examples of meloxicam co-crystals that can be prepared according to the following co-crystallization processes include, but are not limited to:

| Co-crystal | | | Stoichiometry |
|---|---|---|---|
| # | API | Co-former | (API:Co-former) |
| (1) | meloxicam | benzoic acid | 1:1 |
| (2) | meloxicam | acetylsalicylic acid | 1:1 |
| (3) | meloxicam | salicylic acid | 1:1 |
| (4) | meloxicam | 1-hydroxy-2-naphthoic acid | 1:1 |
| (5) | meloxicam | 2,5-dihydroxybenzoic acid | 1:1 |
| (6) | meloxicam | 4-hydroxybenzoic acid | 1:1 |
| (7) | meloxicam | hydrocinnamic acid | 1:1 |

The substantially pure meloxicam co-crystals, herein, may be prepared according to a process comprising forming a solution of meloxicam and a co-former in an organic solvent to form a solution, combining the solution with an anti-solvent, and isolating the meloxicam co-crystal. In certain embodiments, the co-former is an aromatic carboxylic acid, including, but not limited to those in the preceding table. The meloxicam and co-former may be combined in the solution at a molar ratio between about 1:1 and about 1:2. In certain embodiments, the meloxicam:co-former may be combined in the solution at a molar ratio between about 1:1 and about 1:1.5. In certain embodiments, the meloxicam:co-former may be combined in the solution at a molar ratio between about 1:1 and about 1:1.3.

The organic solvent may comprise or consist essentially of a polar aprotic solvent. Suitable organic solvents include, but are not limited to, dimethyl sulfoxide, N,N-dimethylformamide, N,N-dimethylacetamide, N-methylpyrrolidinone, formamide, nitromethane, acetonitrile, dimethyl carbonate, diethyl carbonate, ethylene carbonate, propylene carbonate, or mixtures thereof.

In one embodiment, the organic solvent comprises dimethyl sulfoxide, N,N-dimethylformamide, N,N-dimethylacetamide, N-methylpyrrolidinone, or a mixture thereof. In another embodiment, the solvent comprises dimethyl sulfoxide, N,N-dimethylformamide, or a mixture thereof. In another embodiment, the solvent comprises dimethyl sulfoxide.

In making the meloxicam solution, the organic solvent may be heated to facilitate dissolution of the meloxicam and co-former. For example, the organic solvent may be heated to a temperature up to the boiling point of selected organic solvent or solvent mixture, for an amount of time suitable to form the meloxicam solution. In certain embodiments, the organic solvent may be heated at a temperature between about 40° C. and about 120° C., or between about 50° C. and about 120° C., or between about 60° C. and about 120° C., or between about 70° C. and about 120° C., or between about 80° C. and about 120° C., or between about 90° C. and about 120° C.

In other embodiments, the organic solvent may be heated to a temperature between about 40° C. and about 100° C., or between about 50° C. and about 100° C., or between about 60° C. and about 100° C., or between about 70° C. and about 100° C., or between about 80° C. and about 100° C., between about 90° C. and about 100° C., or about 40° C., or about 50° C., or about 60° C., or about 70° C., or about 75° C., or about 80° C., or about 85° C., or about 90° C., or about 95° C., or about 100° C.

In another embodiment, the organic solvent comprises either (a) dimethyl sulfoxide, N,N-dimethylformamide, N,N-dimethylacetamide, N-methylpyrrolidinone, or a mixture thereof; (b) dimethyl sulfoxide, N,N-dimethylformamide, or a mixture thereof; or (c) dimethyl sulfoxide; and the organic solvent is heated to a temperature up to the boiling point of selected solvent or solvent mixture; or to a temperature between about 50° C. and about 120° C., or between about 70° C. and about 120° C., or between about 80° C. and about 120° C.; or between about 50° C. and about 100° C.; or between about 70° C. and about 100° C.; or between about 80° C. and about 100° C.; or between about 90° C. and about 100° C.; or about 95° C.; or about 100° C.

In another embodiment, the organic solvent comprises either (a) dimethyl sulfoxide, N,N-dimethylformamide, or a mixture thereof; or (b) dimethyl sulfoxide; and the organic solvent is heated to a temperature up to the boiling point of selected organic solvent or solvent mixture; or between about 50° C. and about 120° C., or between about 70° C. and about 120° C., or between about 80° C. and about 120° C.; or between about 50° C. and about 100° C.; or between about 70° C. and about 100° C.; or between about 80° C. and about 100° C.; or between about 90° C. and about 100° C.; or about 95° C.; or about 100° C.

In another embodiment, the organic solvent comprises dimethyl sulfoxide and the organic solvent is heated to a temperature up to the boiling point of organic solvent or solvent mixture; or between about 50° C. and about 120° C., or between about 70° C. and about 120° C., or between about 80° C. and about 120° C.; or between about 50° C. and about 100° C.; or between about 70° C. and about 100° C.; or between about 80° C. and about 100° C.; or between about 90° C. and about 100° C.; or about 95° C.; or about 100° C.

In one example, where the co-former is acetylsalicylic acid, the meloxicam and acetylsalicylic acid may be dissolved in the organic solvent at between about 80° C. or 90° C. to about 100° C. In one embodiment, the meloxicam and co-former may be dissolved in dimethyl sulfoxide or dimethyl formamide at between about 80° C. or 90° C. to about 100° C. In another embodiment, the meloxicam and co-former may be dissolved in dimethyl sulfoxide at about 80° C. or 85° C. or 90° C. or 95° C.

In another example, where the co-former is 1-hydroxy-2-naphthoic acid, the meloxicam and co-former may be dissolved in the organic solvent at between about 80° C. or 90° C. to about 100° C. In one embodiment, the meloxicam and acetylsalicylic acid may be dissolved in dimethyl sulfoxide or dimethyl formamide at between about 80° C. or 90° C. to about 100° C. In another embodiment, the meloxicam and co-former may be dissolved in dimethyl sulfoxide at about 80° C. or 85° C. or 90° C. or 95° C.

In another example, where the co-former is salicylic acid, the meloxicam and co-former may be dissolved in the organic solvent at between about 80° C. or 90° C. to about 100° C. In one embodiment, the meloxicam and acetylsalicylic acid may be dissolved in dimethyl sulfoxide or dimethyl formamide at between about 80° C. or 90° C. to about 100° C. In another embodiment, the meloxicam and co-former may be dissolved in dimethyl sulfoxide at about 80° C. or 85° C. or 90° C. or 95° C.

Following preparation of the solution of meloxicam and the co-former, an antisolvent may be combined with the solution to initiate crystallization of the desired co-crystal. In general, the anti-solvent is a solvent that is miscible with the organic solvent, and in which the desired co-crystal is poorly soluble. In one embodiment, the anti-solvent is water. The order of addition may be either solution to anti-solvent or anti-solvent to solution.

The amount of anti-solvent combined with the organic solution is an amount suitable to encourage crystallization of the desired co-crystal. In certain embodiments, the ratio of organic solvent to anti-solvent is between about 1:99 to 99:1 by volume, such as between 1:50 to 1:1, or 1:50 to 1:3 by volume. In one example, the ratio is about 1:35 or 1:10 or 1:7.5 or 1:5 or 1:3.7 by volume.

Isolating the co-crystal may be done using techniques familiar to those skilled in the art, such as, filtration by gravity or suction, or centrifugation. In one embodiment, the solvent is removed by filtration. The co-crystal, after filtration, may be washed with another solvent. For example, the co-crystal may be washed with a hydrocarbon, such as cyclohexane or n-heptane.

### Co-Crystallization by Slurry Process (Process 2)

Examples of meloxicam co-crystals that can be prepared according to the following slurry processes include, but are not limited to:

| Co-crystal | | | Stoichiometry |
|---|---|---|---|
| # | API | Co-former | (API:Co-former) |
| (1a) | meloxicam | benzoic acid | 1:1 |
| (2a) | meloxicam | acetylsalicylic acid | 1:1 |
| (3a) | meloxicam | salicylic acid | 1:1 |
| (4a) | meloxicam | 1-hydroxy-2-naphthoic acid | 1:1 |
| (5a) | meloxicam | 2,5-dihydroxybenzoic acid | 1:1 |
| (6a) | meloxicam | 4-hydroxybenzoic acid | 1:1 |
| (7a) | meloxicam | hydrocinnamic acid | 1:1 |
| (8a) | meloxicam | succinic acid | 2:1 |
| (9a) | meloxicam | maleic acid | 1:1 |

The substantially pure meloxicam co-crystals, herein, may be prepared according to a process comprising forming a suspension of meloxicam and a co-former in an organic solvent, agitating the suspension (e.g., stirring) for a period of time suitable to provide a meloxicam co-crystal, and isolating the meloxicam co-crystal, wherein either the ratio of meloxicam measured in grams to organic solvent measured in mL (“the w/v ratio”) in the suspension is greater than about 1:5; or one of the meloxicam or the co-former is present in the suspension in at least a 10% molar excess with respect to the other. In one embodiment, the w/v ratio is greater than about 1:5; and one of the meloxicam or the co-former is present in the suspension in at least a 10% molar excess with respect to the other.

The term “molar excess” as used herein means the amount of meloxicam or co-former in the suspension described herein that is in excess of the molar amounts defined by the unit cell of the meloxicam co-crystal in preparation. For example, where the co-crystal is a meloxicam:acetylsalicylic acid co-crystal (1:1), and the suspension contains 1.0 mol of meloxicam and 1.3 mol of acetylsalicylic acid, then the molar excess of acetylsalicylic acid is 30%. In another example, where the co-crystal is a meloxicam:succinic acid co-crystal (2:1), and the suspension contains 0.5 mol of meloxicam and 0.4 mol of succinic acid, then the molar excess of succinic acid is 60%. In the latter example, since meloxicam is limiting, then 0.15 mol more succinic acid is present over the 0.25 mol needed; (0.15 mol excess/0.25 mol needed)×100=60% molar excess of succinic acid.

In one embodiment, the meloxicam is present in at least a 10% molar excess with respect to the co-former. For example, the meloxicam can be present in at least a 15% or 20% or 25% or 30% or 35% or 40% or 45% or 50% or 55% or 60% or 65% or 70% or 75% or 80% or 85% or 90% or 95% or 100% molar excess with respect to the co-former. In other examples, the meloxicam can be present in at least a 10-100% or 10-90% or 10-80% or 10-70% or 10-60% or 10-50% or 10-40% or 10-30% excess with respect to the co-former. In other examples, the meloxicam can be present in at least a 20-100% or 20-90% or 20-80% or 20-70% or 20-60% or 20-50% or 20-40% or 20-30% excess with respect to the co-former.

In another embodiment, the co-former is present in at least a 10% molar excess with respect to the meloxicam. For example, the co-former can be present in at least a 15% or 20% or 25% or 30% or 35% or 40% or 45% or 50% or 55% or 60% or 65% or 70% or 75% or 80% or 85% or 90% or 95% or 100% molar excess with respect to the meloxicam. In other examples, the co-former can be present in at least a 10-100% or 10-90% or 10-80% or 10-70% or 10-60% or 10-50% or 10-40% or 10-30% excess with respect to the meloxicam. In other examples, the co-former can be present in at least a 20-100% or 20-90% or 20-80% or 20-70% or 20-60% or 20-50% or 20-40% or 20-30% excess with respect to the meloxicam. In other examples, the co-former can be present in at least a 30-100% or 30-90% or 30-80% or 30-70% or 30-60% or 30-50% excess with respect to the meloxicam. In other examples, the co-former can be present in at least a 40-100% or 40-90% or 40-80% or 40-70% or 40-60% excess with respect to the meloxicam.

In certain embodiments, the w/v ratio is about 1:5 to about 1:15. In other embodiments, the w/v ratio is about 1:5 to about 1:12; or about 1:5 to about 1:10; or about 1:5 to about 1:8.

In combination, the w/v ratio can be about 1:5 to about 1:15 and the co-former can be present in at least a 10-100% excess or 10-50% excess with respect to the meloxicam. Or, the w/v ratio can be about 1:5 to about 1:10 and the co-former can be present in at least a 10-100% excess or 10-50% excess with respect to the meloxicam. Or, the w/v ratio can be about 1:5 to about 1:8 and the co-former can be present in at least a 10-100% excess or 10-50% excess with respect to the meloxicam.

In another combination, the w/v ratio can be about 1:5 to about 1:15 and the co-former can be present in at least a 20-100% excess or 20-50% excess with respect to the meloxicam. Or, the w/v ratio can be about 1:5 to about 1:10 and the co-former can be present in at least a 20-100% excess or 20-50% excess with respect to the meloxicam. Or, the w/v ratio can be about 1:5 to about 1:8 and the co-former can be present in at least a 20-100% excess or 20-50% excess with respect to the meloxicam.

The organic solvent may comprise or consist essentially of a solvent including, but are not limited to, methanol, ethanol, isopropanol, propanol, butanol, ethyl acetate, isopropyl acetate, acetone, butanone, tetrahydrofuran, dioxane, chloroform, and dichloromethane. In certain embodiments, the organic solvent comprises ethyl acetate, isopropyl acetate, acetone, butanone, tetrahydrofuran, or a mixture thereof. In another embodiment, the organic solvent comprises ethyl acetate, acetone, or tetrahydrofuran. In another embodiment, the organic solvent comprises ethyl acetate.

In making the meloxicam suspension, the organic solvent may be heated. For example, the organic solvent may be heated to a temperature up to 50° C. Additionally, while agitating (e.g., stirring) the meloxicam suspension, the organic solvent may be heated. For example, the organic solvent may be heated to a temperature up to 50° C. In either case, the meloxicam suspension may be maintained at a temperature between about 20° C. and about 50° C. for a period of time suitable to form a meloxicam co-crystal. In certain embodiments, the meloxicam suspension may be maintained at a temperature between about 20° C. and about 40° C.; or about 30° C. and about 40° C., for a period of time suitable to form a meloxicam co-crystal. In each of the preceding embodiments, a suitable period of time may be between about 5 minutes and about 48 hours; or between about 1 hour and 48 hours; or between about 2 hours and 48 hours; or between about 4 hours and 48 hours; or between about 8 hours and 48 hours; or between about 12 hours and 48 hours; or between about 4 hours and 24 hours; or between about 8 hours and 24 hours; or between about 12 hours and 24 hours.

After the initial period of time, a second volume of solvent organic solvent may be optionally added to the preceding suspension. The second volume may be provided at a w/v ratio of about 1:1 to about 1:5 in addition to the volume present in the meloxicam suspension. In other embodiments, the w/v ratio of the second volume is about 1:1 to about 1:3; or about 1:2 w/v. The meloxicam suspension may be stirred for an additional period of time may be between about 5 minutes and about 12 hours; or between about 1 hour and 8 hours; or between about 1 hours and 6 hours; or between about 1 hour and 4 hours; or between about 2 hours and 4 hours. During this additional period of time, the temperature of the meloxicam suspension may be maintained at the same temperature as previous.

In one example, where the co-former is maleic acid, the meloxicam and co-former may be suspended in the organic solvent at between about 20° C. to about 40° C. In one embodiment, the meloxicam and maleic acid may be suspended in ethyl acetate at between about 30° C. to about 40° C.

In certain embodiments, the w/v ratio is between about 1:5 to 1:10, such as between 1:5 and 1:8. In certain embodiments, the co-former is present in about 10-100% molar excess, such as about 30% molar excess (e.g., meloxicam:maleic acid, 1 mol:1.3 mol).

In another example, where the co-former is succinic acid, the meloxicam and co-former may be suspended in the organic solvent at between about 20° C. to about 40° C. In one embodiment, the meloxicam and succinic acid may be suspended in ethyl acetate at between about 30° C. to about 40° C. In certain embodiments, the w/v ratio is between about 1:5 to 1:10, such as 1:5 and 1:8. In certain embodiments, the co-former is present in about 10-100% molar excess, such as about 30% molar excess, or about 10-50% molar excess, or about 10-35% molar excess, or about 10-25% molar excess, or about 11% molar excess (e.g., meloxicam:succinic acid, 1 mol:0.55 mol).

In another example, where the co-former is acetylsalicylic acid, the meloxicam and co-former may be suspended in the organic solvent at between about 20° C. to about 40° C. In one embodiment, the meloxicam and acetylsalicylic acid may be suspended in ethyl acetate at between about 30° C. to about 40° C. In certain embodiments, the w/v ratio is between about 1:5 and 1:10, such as about 1:5 to 1:8. In certain embodiments, the co-former is present in about 10-100% molar excess, such as about 20%, or about 30% or about 40% or about 50% or about 60% molar excess (e.g., meloxicam:acetylsalicylic acid, 1 mol:1.2 mol; or meloxicam:acetylsalicylic acid, 1 mol:1.4 mol).

In another example, where the co-former is salicylic acid, the meloxicam and co-former may be suspended in the organic solvent at between about 20° C. to about 40° C. In one embodiment, the meloxicam and acetylsalicylic acid may be suspended in ethyl acetate at between about 30° C. to about 40° C. In certain embodiments, the w/v ratio is between about 1:5 and 1:10, such as between about 1:5 and 1:8. In certain embodiments, the molar ratio of meloxicam to co-former is between about 1:1 to 1:2. In certain embodiments, the co-former is present in about 10-100% molar excess, such as about 30%, or about 40%, or about 50%, or about 60% molar excess (e.g., meloxicam:salicylic acid, 1 mol:1.3 mol; or meloxicam:salicylic acid, 1 mol:1.5 mol).

Isolating the co-crystal may be done using techniques familiar to those skilled in the art, such as, filtration by gravity or suction, or centrifugation. In one embodiment, the solvent is removed by filtration. The resulting solids may be optionally washed with a suitable volume of an organic solvent, as defined above. In particular, the resulting solids may be optionally washed with a suitable volume of the same organic solvent as used for the suspension process above.

In certain embodiments, the meloxicam co-crystal may be contacted with water for a suitable period of time to remove at least a portion of a water-soluble impurity, that is, to provide a meloxicam co-crystal having less than about 0.5 wt. %, or less than about 0.3 wt. %, or less than about 0.15 wt. %, or less than about 0.10 wt. %, or less than about 0.05 wt. % of a water-soluble impurity. Such contacting may occur at a temperature between about 20° C. and about 100° C., such as between about 30° C. and about 75° C., or between about 40° C. and about 60° C., or about 50° C. In certain embodiments, the water-soluble impurity can comprise free co-former. In one embodiment, the free co-former is succinic acid, fumaric acid, or maleic acid. In another embodiment, the free co-former is succinic acid.

### Co-Crystal Characterization

As is familiar to those skilled in the art, impurity characterization and quantification levels can be a function of the method utilized. For example, HPLC methods can detect impurities at about 0.05 wt. % quantities. However, in certain embodiments, quantification of residual impurities (e.g., free meloxicam and/or co-former) by PXRD methods may be limited by detector sensitivity and corresponding noise factors involved in extended PXRD scans. PXRD methods can detect less than about 1.0 wt. % quantities. In certain embodiments, PXRD methods may detect less than about 0.3 wt. % or about 0.1 wt. % quantities.

### Powder X-Ray Diffraction Analysis of Meloxicam Co-Crystals (PXRD)

The co-crystals of the present disclosure can be characterized by their powder X-ray diffraction patterns. Thus, the X-ray diffraction patterns of the co-crystals of provided herein were measured on a PANalytical X'PertPRO Powder X-ray diffractometer equipped with goniometer of θ/θ configuration and X'celerator detector. The Cu-anode X-ray tube was operated at 45 kV and 40 mA. The experiments were conducted over the 2θ range of 2.0°-50.0°, 0.030° step size and 189.865 time per step (seconds) and all the co-crystals were characterized with above mentioned method.

### Analysis of Residual Solvents by GC-HS

The residual solvent levels in meloxicam co-crystals were determined by using gas chromatography: Agilent Technologies, 6890 N system equipped with flame ionization detector or its equivalent.
Chemicals: Ethyl acetate, n-heptane, dimethyl sulfoxide, N-methyl pyrrolidone.
Head space sampler: Agilent Technologies, G1888 or its equivalent.
Data handling system: Empower software or its equivalent.
Column: CP select-624 capillary column with 60 mts length, 0.32 mm internal diameter and 1.8 micron film thickness or its equivalent.
Run time: 33 minutes.

### HPLC Meloxicam Co-Crystal Assay

Instrumentation: Waters HPLC system having 2695 model pump having UV/PDA detector with Empower chromatography software or its equivalent.
Column: (A) Unison UK C18 column; (B) YMC Trait C18 EXRS Column, 250×4.6 mm, 5.0 micron; (C) Water Atlantis T3, 250×4.6 mm, 5 micron.
Mobile Phase: (A) methanol-acetonitrile-dipotassium phosphate buffer; or (B) acetonitrile-Sodium sulfate buffer solution
Detection wavelength: 210 nm and 360 nm

| | | | Flow Rate |
|---|---|---|---|
| Entity | Column | Mobile Phase | (mL/min) |
| Meloxicam | B | A | 1.0 |
| Acetylsalicylic acid | B | A | 1.0 |
| Salicylic Acid | B | A | 1.0 |
| Maleic Acid | B | A | 1.0 |
| 1-Hydroxy-2-Naphthoic acid | A | A | 0.5 |
| Succinic Acid | C | B | 0.8 |

### Analysis of Impurities by HPLC (Purity Assay)

The HPLC method was established for determination of related substances (e.g., impurities) of meloxicam co-crystals. A Zorbax Eclipse XDB C18 column was used with the mobile phase of methanol-acetonitrile-10 mM phosphate buffer solution at the detection wavelength of 350 nm and 260 nm.
Instrumentation: Waters HPLC system having 2695 model pump having UV/PDA detector with Empower chromatography software or its equivalent.
Chemicals: Dipotassium hydrogen phosphate, orthophosphoric acid, acetonitrile, water, sodium hydroxide, methanol.
Column: Zorbax Eclipse XDB C18 column, 250×4.6 mm, 5 μm.
Elution: Gradient.
Flow rate: 0.8 mL/min.

### EXEMPLARY EMBODIMENTS

[Embodiment 1] A substantially pure co-crystal of meloxicam with a co-former.

[Embodiment 2] The co-crystal of [Embodiment 1], wherein the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, and salicylic acid.

[Embodiment 3] The co-crystal of [Embodiment 1], wherein the co-former is 1-hydroxy-2-naphthoic acid.

[Embodiment 4] The co-crystal of [Embodiment 1], wherein the co-former is acetylsalicylic acid.

[Embodiment 5] The co-crystal of [Embodiment 1], wherein the co-former is benzoic acid.

[Embodiment 6] The co-crystal of [Embodiment 1], wherein the co-former is 2,5-dihydroxybenzoic acid.

[Embodiment 7] The co-crystal of [Embodiment 1], wherein the co-former is 4-hydroxybenzoic acid.

[Embodiment 8] The co-crystal of [Embodiment 1], wherein the co-former is hydrocinnamic acid.

[Embodiment 9] The co-crystal of [Embodiment 1], wherein the co-former is salicylic acid.

[Embodiment 10] The co-crystal of any one of [Embodiments 1-9], having less than about 2 wt. % total impurities.

[Embodiment 11] The co-crystal of any one of [Embodiments 1-9], having less than about 1 wt. % total impurities.

[Embodiment 12] The co-crystal of any one of [Embodiments 1-9], having less than about 0.5 wt. % total impurities.

[Embodiment 13] The co-crystal of any one of [Embodiments 1-9], having less than about 0.3 wt. % total impurities.

[Embodiment 14] The co-crystal of any one of [Embodiments 10-12], having less than about 0.5 wt. % of any one impurity.

[Embodiment 15] The co-crystal of any one of [Embodiments 10-13], having less than about 0.3 wt. % of any one impurity.

[Embodiment 16] The co-crystal of any one of [Embodiments 10-13], having less than about 0.15 wt. % of any one impurity.

[Embodiment 17] The co-crystal of any one of [Embodiments 10-13], having less than about 0.10 wt. % of any one impurity.

[Embodiment 18] The co-crystal of any one of [Embodiments 10-13], having less than about 0.05 wt. % of any one impurity.

[Embodiment 19] The co-crystal of any one of [Embodiments 14-18], wherein the impurity is free meloxicam.

[Embodiment 20] The co-crystal of any one of [Embodiments 14-18], wherein the impurity is free co-former.

[Embodiment 21] The co-crystal of any one of [Embodiments 1-20] having less than about 5000 ppm of any one residual solvent.

[Embodiment 22] The co-crystal of any one of [Embodiments 1-20] having less than about 4000 ppm of any one residual solvent.

[Embodiment 23] The co-crystal of any one of [Embodiments 1-20] having less than about 3000 ppm of any one residual solvent.

[Embodiment 24] The co-crystal of any one of [Embodiments 1-20] having less than about 2000 ppm of any one residual solvent.

[Embodiment 25] The co-crystal of any one of [Embodiments 1-20] having less than about 1000 ppm of any one residual solvent.

[Embodiment 26] The co-crystal of any one of [Embodiments 1-20] having less than about 500 ppm of any one residual solvent.

[Embodiment 27] The co-crystal of any one of [Embodiments 21-26], wherein the residual solvent is dimethyl sulfoxide and/or N,N-dimethylformamide.

[Embodiment 28] The co-crystal of any one of [Embodiments 21-26], wherein the residual solvent is dimethyl sulfoxide.

[Embodiment 29] A process for preparing a meloxicam co-crystal comprising: combining a solution comprising meloxicam, a co-former, and an organic solvent, with an anti-solvent; and isolating the meloxicam co-crystal.

[Embodiment 30] The process of [Embodiment 29], wherein the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, and salicylic acid.

[Embodiment 31] The process of [Embodiment 29], wherein the co-former is 1-hydroxy-2-naphthoic acid.

[Embodiment 32] The process of [Embodiment 29], wherein the co-former is acetylsalicylic acid.

[Embodiment 33] The process of [Embodiment 29], wherein the co-former is benzoic acid.

[Embodiment 34] The process of [Embodiment 29], wherein the co-former is 2,5-dihydroxybenzoic acid.

[Embodiment 35] The process of [Embodiment 29], wherein the co-former is 4-hydroxybenzoic acid.

[Embodiment 36] The process of [Embodiment 29], wherein the co-former is hydrocinnamic acid.

[Embodiment 37] The process of [Embodiment 29], wherein the co-former is salicylic acid.

[Embodiment 38] The process of any one of [Embodiments 29-37], wherein the organic solvent comprises or consists essentially of a polar aprotic solvent.

[Embodiment 39] The process of [Embodiment 38], wherein the organic solvent comprises or consists essentially of dimethyl sulfoxide, N,N-dimethylformamide, N,N-dimethylacetamide, N-methylpyrrolidinone, formamide, nitromethane, acetonitrile, dimethyl carbonate, diethyl carbonate, ethylene carbonate, propylene carbonate, or a mixture thereof.

[Embodiment 40] The process of [Embodiment 38], wherein the organic solvent comprises or consists essentially of dimethyl sulfoxide, N,N-dimethylformamide, N,N-dimethylacetamide, N-methylpyrrolidinone, or a mixture thereof.

[Embodiment 41] The process of [Embodiment 38], wherein the organic solvent comprises or consists essentially of dimethyl sulfoxide, N,N-dimethylformamide, or a mixture thereof.

[Embodiment 42] The process of [Embodiment 38, wherein the organic solvent comprises or consists essentially of dimethyl sulfoxide.

[Embodiment 43] The process of any one of [Embodiments 29-42], further comprising forming the solution by heating the organic solvent, meloxicam, and co-former.

[Embodiment 44] The process of any one of [Embodiments 29-42], further comprising forming the solution by heating the organic solvent, meloxicam, and co-former to a temperature between about 40° C. and about 120° C.

[Embodiment 45] The process of any one of [Embodiments 29-42], further comprising forming the solution by heating the organic solvent, meloxicam, and co-former to a temperature between about 70° C. and about 100° C.

[Embodiment 46] The process of any one of [Embodiments 29-42], further comprising forming the solution by heating the organic solvent, meloxicam, and co-former to a temperature between about 90° C. and about 100° C.

[Embodiment 47] The process of any one of [Embodiments 29-46], wherein the isolating is filtration or centrifugation.

[Embodiment 48] A substantially pure co-crystal of meloxicam with salicylic acid.

[Embodiment 49] A substantially pure co-crystal of meloxicam with acetylsalicylic acid.

[Embodiment 50] A substantially pure co-crystal of meloxicam with 1-hydroxy-2-naphthoic acid.

[Embodiment 51] The substantially pure co-crystal of [Embodiments 48-50], comprising between 100 ppm and 5000 ppm of any one residual solvent.

[Embodiment 52] The substantially pure co-crystal of [Embodiment 51], wherein the residual solvent is dimethyl sulfoxide.

[Embodiment 53] The substantially pure co-crystal of any one of [Embodiments 48-52], having less than about 2 wt. % total impurities.

[Embodiment 54] The substantially pure co-crystal of any one of [Embodiments 48-52], having less than about 1 wt. % total impurities.

[Embodiment 55] The substantially pure co-crystal of any one of [Embodiments 48-52], having less than about 0.5 wt. % total impurities.

[Embodiment 56] The substantially pure co-crystal of any one of [Embodiments 48-52], having less than about 0.3 wt. % total impurities.

[Embodiment 57] The substantially pure co-crystal of any one of [Embodiments 48-52], having less than about 0.5 wt. % of any one impurity.

[Embodiment 58] The substantially pure co-crystal of any one of [Embodiments 48-52], having less than about 0.3 wt. % of any one impurity.

[Embodiment 59] The substantially pure co-crystal of any one of [Embodiments 48-52], having less than about 0.15 wt. % of any one impurity.

[Embodiment 60] The substantially pure co-crystal of any one of [Embodiments 48-52], having less than about 0.10 wt. % of any one impurity.

[Embodiment 61] The substantially pure co-crystal of any one of [Embodiments 48-52], having less than about 0.05 wt. % of any one impurity.

[Embodiment 62] The substantially pure co-crystal of any one of [Embodiments 57-61], wherein the impurity is free meloxicam.

[Embodiment 63] The co-crystal of any one of [Embodiments 1-9], having less than about 1 wt. % free meloxicam.

[Embodiment 64] The co-crystal of any one of [Embodiments 1-9], characterized by an absence of one or more PXRD signals attributable to free meloxicam selected from the group consisting of 6.5, 11.2, 13.2, 14.9, and 17.8°+1-0.2° 20.

[Embodiment 65] The co-crystal of [Embodiment 3 (1-hydroxy-2-naphthoic acid co-former)], characterized by an absence of the PXRD signal attributable to free meloxicam at 13.2°+/−0.2° 20.

[Embodiment 66] The co-crystal of [Embodiment 4 (acetylsalicylic acid)], characterized by an absence of the PXRD signal attributable to free meloxicam at 11.2°+1-0.2° 20.

[Embodiment 67] The co-crystal of [Embodiment 5 (salicylic acid)], characterized by an absence of the PXRD signal attributable to free meloxicam at 6.5°+1-0.2° 20. [Embodiment 1a] A substantially pure co-crystal of meloxicam with a co-former.

[Embodiment 2a] The co-crystal of [Embodiment 1a], wherein the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, succinic acid, maleic acid, salicylic acid, fumaric acid, adipic acid, DL-malic acid, L-malic acid, glutaric acid, malonic acid, glycolic acid, camphoric acid, maltol, and ethyl maltol.

[Embodiment 3a] The co-crystal of [Embodiment 1a], wherein the co-former is succinic acid.

[Embodiment 4a] The co-crystal of [Embodiment 1a], wherein the co-former is maleic acid.

[Embodiment 5a] The co-crystal of any one of [Embodiments 1a-4a], having less than about 2 wt. % total impurities.

[Embodiment 6a] The co-crystal of any one of [Embodiments 1a-4a], having less than about 1 wt. % total impurities.

[Embodiment 7a] The co-crystal of any one of [Embodiments 1a-4a], having less than about 0.5 wt. % total impurities.

[Embodiment 8a] The co-crystal of any one of [Embodiments 1a-14a] having less than about 0.3 wt. % total impurities.

[Embodiment 9a] The co-crystal of any one of [Embodiments 6a-8a], having less than about 0.5 wt. % of any one impurity.

[Embodiment 10a] The co-crystal of any one of [Embodiments 6a-9a], having less than about 0.3 wt. % of any one impurity.

[Embodiment 11a] The co-crystal of any one of [Embodiments 6a-9a], having less than about 0.15 wt. % of any one impurity.

[Embodiment 12a] The co-crystal of any one of [Embodiments 6a-9a], having less than about 0.10 wt. % of any one impurity.

[Embodiment 13a] The co-crystal of any one of [Embodiments 6a-9a], having less than about 0.05 wt. % of any one impurity.

[Embodiment 14a] The co-crystal of any one of [Embodiments 9a-12a], wherein the impurity is free meloxicam.

[Embodiment 15a] The co-crystal of any one of [Embodiments 9a-12a], wherein the impurity is free co-former.

[Embodiment 16a] A process for preparing a meloxicam co-crystal comprising: forming a suspension of meloxicam and a co-former in an organic solvent, agitating the suspension (e.g., stirring) for a period of time suitable to provide a meloxicam co-crystal, and isolating the meloxicam co-crystal, wherein the ratio of meloxicam measured in grams to organic solvent measured in mL (“the w/v ratio”) in the suspension is greater than about 1:5; and either the meloxicam or the co-former is present in the suspension in at least a 10% molar excess with respect to the other.

[Embodiment 17a] The process of [Embodiment 16a], wherein the w/v ratio is greater than about 1:5; and one of the meloxicam or the co-former is present in the suspension in at least a 10% molar excess with respect to the other.

[Embodiment 18a] The process of [Embodiment 16a or 17a], wherein the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, succinic acid, maleic acid, salicylic acid, fumaric acid, adipic acid, DL-malic acid, L-malic acid, glutaric acid, malonic acid, glycolic acid, camphoric acid, maltol, and ethyl maltol.

[Embodiment 19a] The process of [Embodiment 16a or 17a], wherein the co-former is 1-hydroxy-2-naphthoic acid.

[Embodiment 20a] The process of [Embodiment 16a or 17a], wherein the co-former is acetylsalicylic acid.

[Embodiment 21a] The process of [Embodiment 16a or 17a], wherein the co-former is salicylic acid.

[Embodiment 22a] The process of [Embodiment 16a or 17a], wherein the co-former is succinic acid.

[Embodiment 23a] The process of [Embodiment 16a or 17a], wherein the co-former is maleic acid.

[Embodiment 24a] The process of any one of [Embodiments 16a-23a], wherein the isolating is filtration or centrifugation.

[Embodiment 25a] The process of any one of [Embodiments 16a-24a], wherein the w/v ratio between about 1:5 and 1:15.

[Embodiment 26a] The process of any one of [Embodiments 16a-25a], wherein the co-former is present in the suspension in at least a 10% molar excess with respect to the meloxicam.

[Embodiment 27a] The process of any one of [Embodiments 16a-25a], wherein the co-former is present in the suspension in about 10%-100% molar excess with respect to the meloxicam.

[Embodiment 28a] The process of any one of [Embodiments 16a-27a], further comprising contacting the meloxicam co-crystal with water for a suitable period of time to remove at least a portion of a water-soluble impurity.

[Embodiment 29a] The process of [Embodiment 28a], wherein the impurity is free co-former.

[Embodiment 30a] The process of [Embodiment 29a], wherein the co-former is succinic acid, fumaric acid, or maleic acid.

[Embodiment 31a] The process of [Embodiment 30a], wherein the co-former is succinic acid.

[Embodiment 32a] A substantially pure co-crystal of meloxicam with succinic acid.

[Embodiment 33a] A substantially pure co-crystal of meloxicam with maleic acid.

[Embodiment 34a] The substantially pure co-crystal of any one of [Embodiments 32a-33a], having less than about 2 wt. % total impurities.

[Embodiment 35a] The substantially pure co-crystal of any one of [Embodiments 32a-33a], having less than about 1 wt. % total impurities.

[Embodiment 36a] The substantially pure co-crystal of any one of [Embodiments 32a-33a], having less than about 0.5 wt. % total impurities.

[Embodiment 37a] The substantially pure co-crystal of any one of [Embodiments 32a-33a], having less than about 0.3 wt. % total impurities.

[Embodiment 38a] The substantially pure co-crystal of any one of [Embodiments 32a-35a], having less than about 0.5 wt. % of any one impurity.

[Embodiment 39a] The substantially pure co-crystal of any one of [Embodiments 32a-36a], having less than about 0.3 wt. % of any one impurity.

[Embodiment 40a] The substantially pure co-crystal of any one of [Embodiments 32a-37a], having less than about 0.15 wt. % of any one impurity.

[Embodiment 41a] The substantially pure co-crystal of any one of [Embodiments 32a-37a], having less than about 0.10 wt. % of any one impurity.

[Embodiment 42a] The substantially pure co-crystal of any one of [Embodiments 32a-37a], having less than about 0.05 wt. % of any one impurity.

[Embodiment 43a] The co-crystal of any one of [Embodiments 32a-42a], wherein the impurity is free meloxicam.

[Embodiment 44a] The co-crystal of any one of [Embodiments 32a-42a], wherein the impurity is free co-former.

### EXAMPLES

### Example 1. Preparation of Co-Crystal Meloxicam:Acetylsalicylic Acid (1:1) Via Process 1

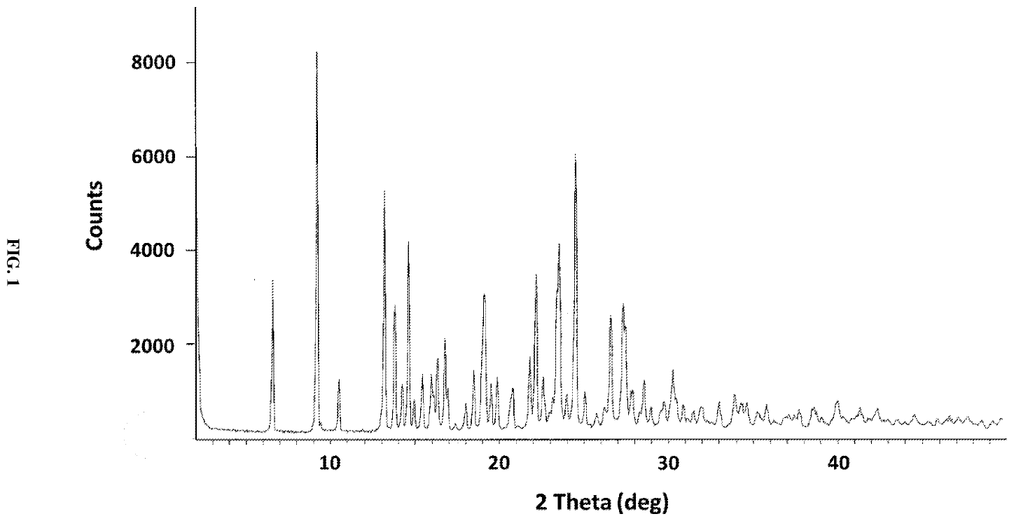

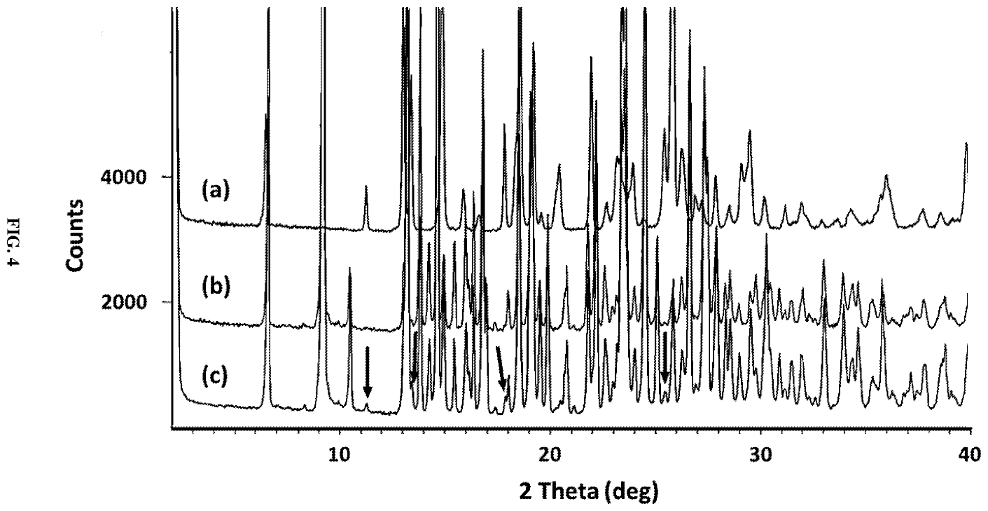

Meloxicam (50 g, 0.142 mol) and acetylsalicylic acid (30.76 g, 0.171 mol) were dissolved in dimethyl sulfoxide (300 mL) at 90±5° C. The clear solution was filtered through a HYFLO bed at 90±5° C. The particle free solution was added to water (1000 mL) at 25±5° C. and maintained at that temperature with stirring at 200 rpm for 15-30 minutes. The temperature was raised to 40-50° C. and slowly cooled to 25±5° C. over 1-2 hours. The reaction mass was maintained under stirring for 15-16 hours at same temperature. The reaction mass was filtered and washed with water (50 mL×2) and dried on filter for 30-60 minutes. The obtained product was dried at 55±5° C. under vacuum for 24-48 hours. The obtained solid (72.0 g) was checked by PXRD (FIG. 1) and identified as co-crystal meloxicam:acetylsalicylic acid (1:1). A comparison of the improved purity of the co-crystal as compared to the prior art process of U.S. Pat. No. 8,124,603 is illustrated in FIG. 4. Meloxicam contamination can be seen by the presence of characteristic peaks at about 11.3, 13.4, 17.9, 25.4(°) 2Θ as indicated by the arrows.

### Example 2. Preparation of Co-Crystal Meloxicam:1-Hydroxy-2-Naphthoic Acid (1:1) Via Process 1

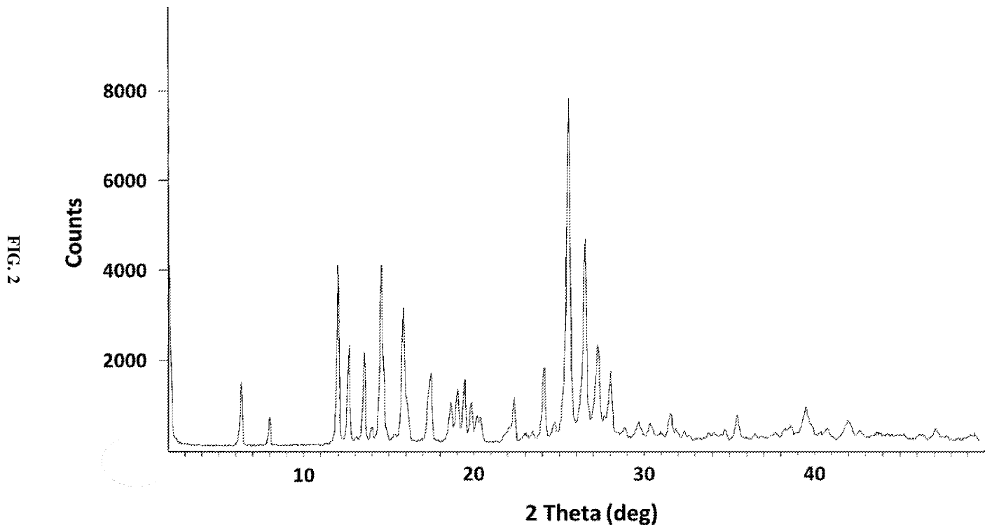

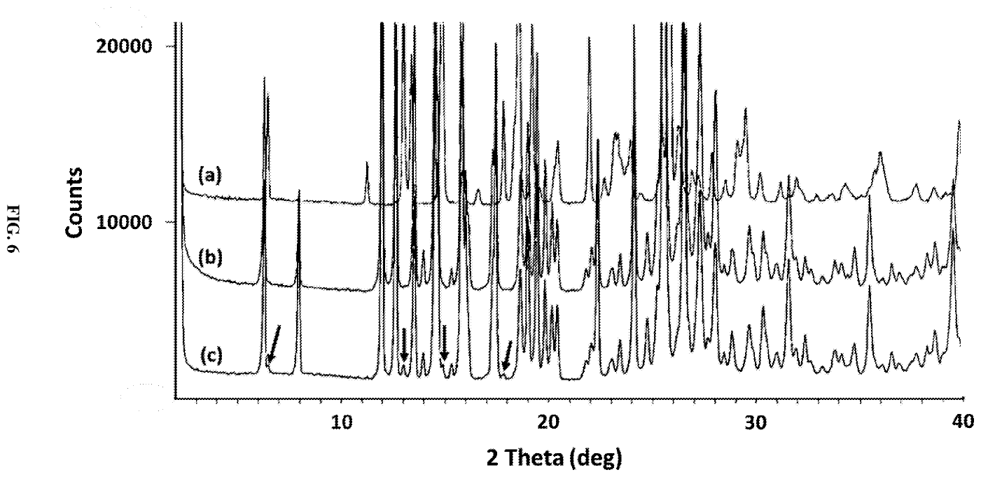

Meloxicam (50 g, 0.142 mol) and 1-hydroxy-2-naphthoic acid (26.76 g, 0.142 mol) were dissolved in dimethyl sulfoxide (300 mL) at 90±5° C. The clear solution was filtered through a HYFLO bed at 90±5° C. The particle free solution was added to water (1000 mL) at 25±5° C. and maintained at that temperature with stirring at 200 rpm for 15-30 minutes. The temperature was raised to 40-50° C. and slowly cooled to 25±5° C. over 1-2 hours. The reaction mass was maintained under stirring for 15-16 hours at the same temperature. The reaction mass was filtered and washed with water (50 mL×2) and dried on the filter for 30-60 minutes. The obtained product was dried at 55±5° C. under vacuum for 24-48 hours. The obtained solid (71.0 g) was checked by PXRD (FIG. 2) and identified as co-crystal meloxicam:1-hydroxy-2-naphthoic acid (1:1). A comparison of the improved purity of the co-crystal as compared to the prior art process of U.S. Pat. No. 8,124,603 is illustrated in FIG. 6. Meloxicam contamination can be seen by the presence of characteristic peaks at 6.5, 13.0, 14.9, 17.8(°) 2Θ as indicated by the arrows.

### Example 3. Preparation of Co-Crystal Meloxicam:Salicylic Acid Form III (1:1) Via Process 1

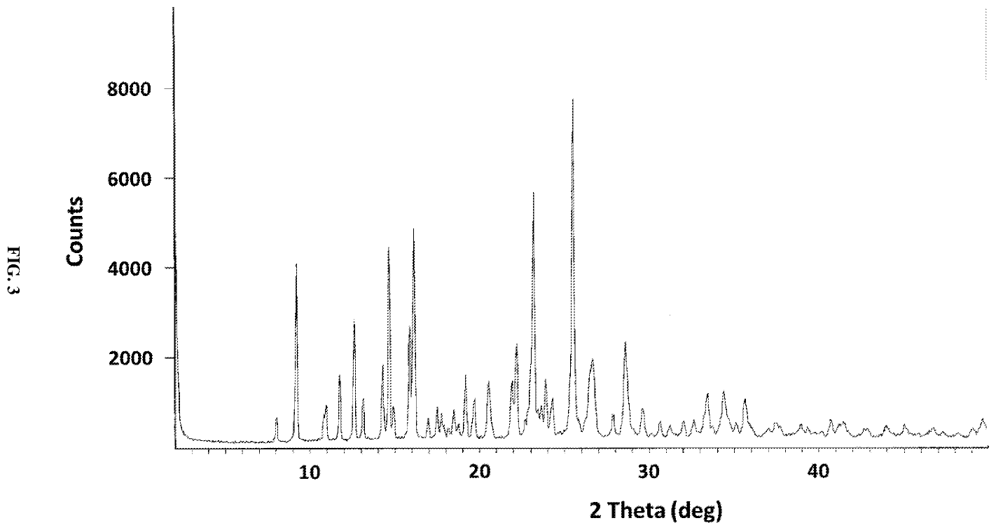

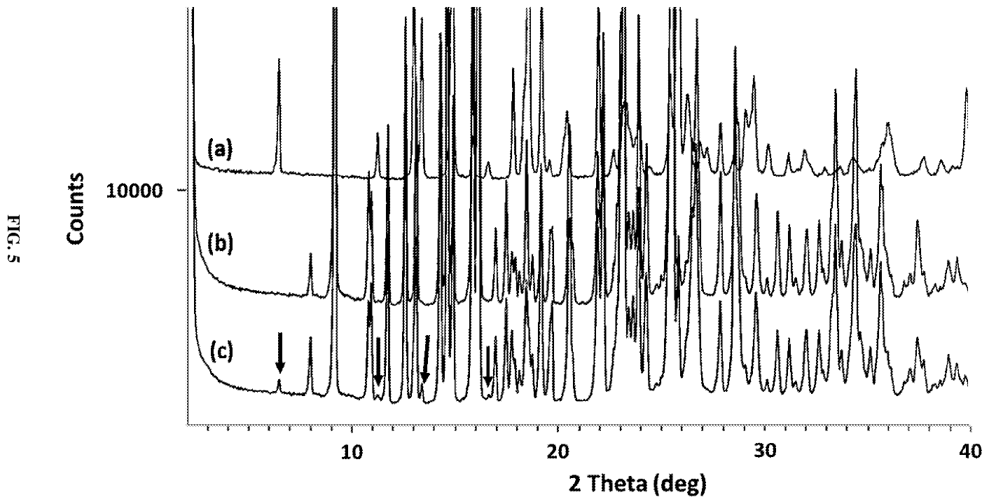

Meloxicam (50 g, 0.142 mol) and salicylic acid (25.54 g, 0.185 mol) were dissolved in dimethyl sulfoxide (300 mL) at 90±5° C. The clear solution was filtered through a HYFLO bed at 90±5° C. The particle free solution was added to water (1000 mL) at 25±5° C. and maintained at that temperature with stirring at 200 rpm for 15-30 minutes. The temperature was raised to 40-50° C. and slowly cooled to 25±5° C. over 1-2 hours. The reaction mass was maintained under stirring for 15-16 hours at same temperature. The reaction mass was filtered and washed with water (50 mL×2) and dried on the filter for 30-60 minutes. The obtained product was dried at 55±5° C. under vacuum for 24-48 hours. The obtained solid (65.5 g) was checked by PXRD (FIG. 3) and identified as co-crystal meloxicam:salicylic acid Form III (1:1). A comparison of the improved purity of the co-crystal as compared to the prior art process of U.S. Pat. No. 8,124,603 is illustrated in FIG. 5. Meloxicam contamination can be seen by the presence of characteristic peaks at 6.5, 11.3, 13.4, 16.6(°) 2Θ as indicated by the arrows.

### Example 4. Residual Solvents in Co-Crystals Prepared by Prior Art Process

Various meloxicam co-crystals were prepared according to the processes of Examples 42-46 in U.S. Pat. No. 8,124,603. The co-crystals were subjected to drying at 40° C. under vacuum for 24 h and residual solvent levels in co-crystals were determined by GC analysis. Results are given in Table 1. It can be noted that the residual solvent levels in the 1-hydroxy-2-naphthoic acid co-crystal exceed ICH limits (<5000 ppm), being in the range of 6000-10,000 ppm even after extended hours of drying up to 72 h. The residual solvent content for the 1-hydroxy-2-naphthoic acid co-crystal increased with increasing batch size.

TABLE 1

| | | Ethyl acetate content |
|---|---|---|
| Co-former | Batch Size | by GC (ppm) |
| Acetylsalicylic acid | 70 g | 1877 |
| 1-hydroxy-2-naphthoic acid | 70 g | 6747 |
| 1-hydroxy-2-naphthoic acid | 150 g | 9679 |
| Salicylic acid | 70 g | 2125 |

### Example 5. Residual Solvents in Co-Crystals Prepared by Examples 1-3

Various meloxicam co-crystals were prepared according to the processes of Examples 1-3 and residual solvent levels in co-crystals were determined by gas chromatography (GC). Results are given in Table 2.

TABLE 2

| | | DMSO content |
|---|---|---|
| Co-former | Batch Size | by GC (ppm) |
| Acetylsalicylic acid | 70 g | 554 |
| 1-hydroxy-2-naphthoic acid | 70 g | 492 |
| Salicylic acid | 70 g | 865 |

### Example 6. Preparation of Co-Crystal Meloxicam:1-Hydroxy-2-Naphthoic Acid (1:1) Via Process 1

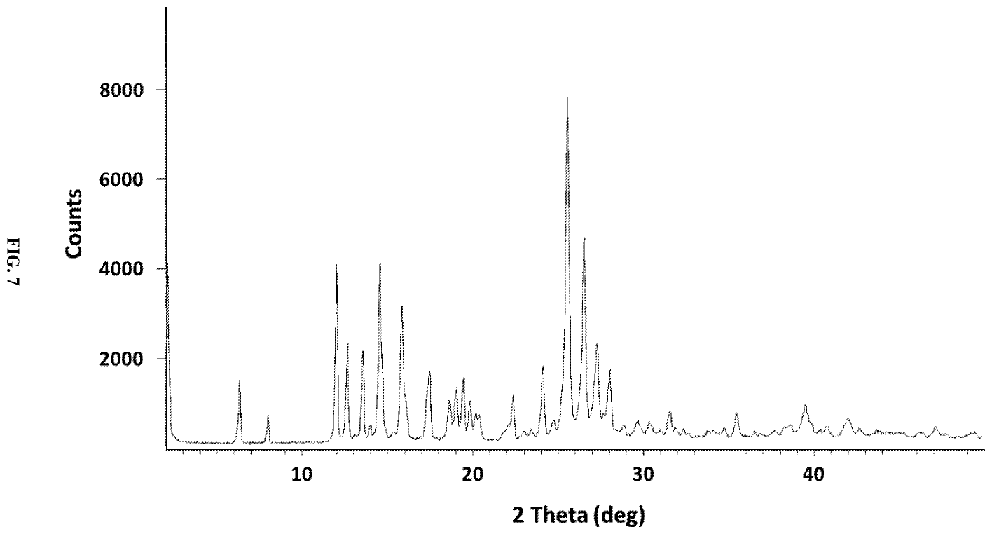

Meloxicam (3.5 kg, 9.96 mol) and 1-hydroxy-2-naphthoic acid (1.872 kg, 9.95 mol) were dissolved in dimethyl sulfoxide (2 L) at 80±5° C. The clear solution was filtered through a 0.2 μm filter at 80±5° C. The particle free solution was added to water (70 L) at 30±5° C. with stirring at 110 rpm for 15-30 minutes, during the addition temperature raise was observed up to 40-50° C. and slowly cooled to 25±5° C. in 1-2 hours. The reaction mass was maintained under stirring for 15-16 hours at 30±5° C. The reaction mass was filtered and washed with water (7 L). Suck dried the material thoroughly for 30 mins. Wet cake washed with n-heptane (2×3.5 L). Suck dried the material for 120 minutes at 30±5° C. Solid material was vacuum dried for 150±30 minutes at 30±5° C. and then then dried at 55±5° C. under vacuum for 18 hours. Cooled the material to below 35° C. and mill the material at 30±5° C. with 1 mm mesh. Milled material was further dried under vacuum at 55±5° C. for 16 hours. The obtained solid (4.81 kg) was checked by PXRD (FIG. 7) and identified as co-crystal meloxicam:1-hydroxy-2-naphthoic acid (1:1).

### Example 7. Preparation of Co-Crystal Meloxicam:1-Hydroxy-2-Naphthoic Acid (1:1) Via Process 1

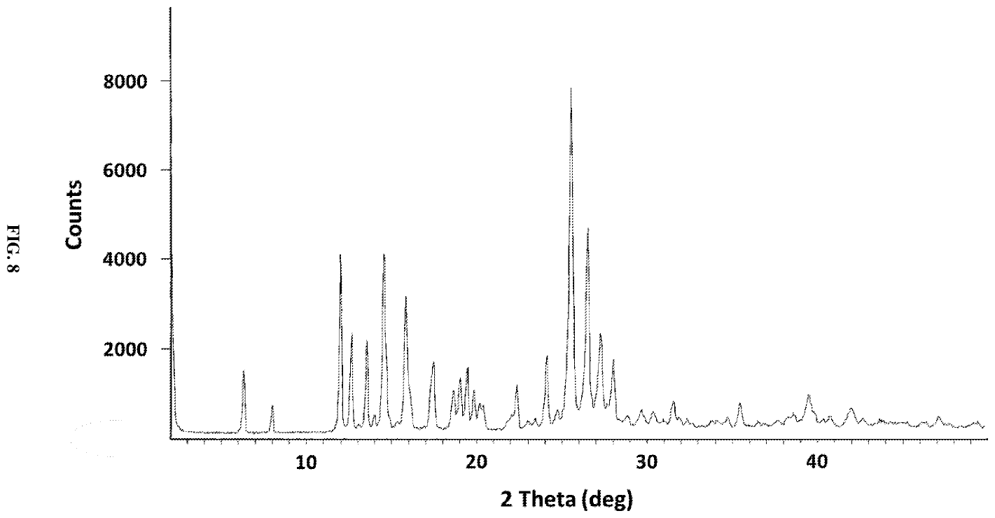

Meloxicam (100 g, 0.285 mol) and 1-hydroxy-2-naphthoic acid (53.44 g, 0.284 mol) were dissolved in dimethyl sulfoxide (550 mL) at 80±5° C. The clear solution was filtered through a HYFLO at 80±5° C. The particle free solution was added to water (2000 mL) at 30±5° C. with stirring at 200 rpm for 15-30 minutes, during the addition temperature raise was observed up to 40-50° C. and slowly cooled to 25±5° C. in 1-2 hours. The reaction mass was maintained under stirring for 15-16 hours at 30±5° C. The reaction mass was then filtered, washed with water (50 mL×2) and suck dried the material thoroughly for 30 minutes. Wet cake was washed with n-heptane (100 mL). Suck dried the material for 120 minutes at 30±5° C. and then vacuum dried for 150±30 minutes at 30±5° C. The solid material was then dried at 57±3° C. under vacuum for 18 hours. Cooled the material to below 35° C. Mill the material at 30±5° C. with 1 mm mesh. Milled material further dried under vacuum at 57±3° C. for 16 hours. The obtained solid (71.0 g) was checked by PXRD (FIG. 8) and identified as co-crystal meloxicam:1-hydroxy-2-naphthoic acid (1:1).

### Example 8. Preparation of Co-Crystal Meloxicam:Acetylsalicylic Acid (1:1) Via Process 2

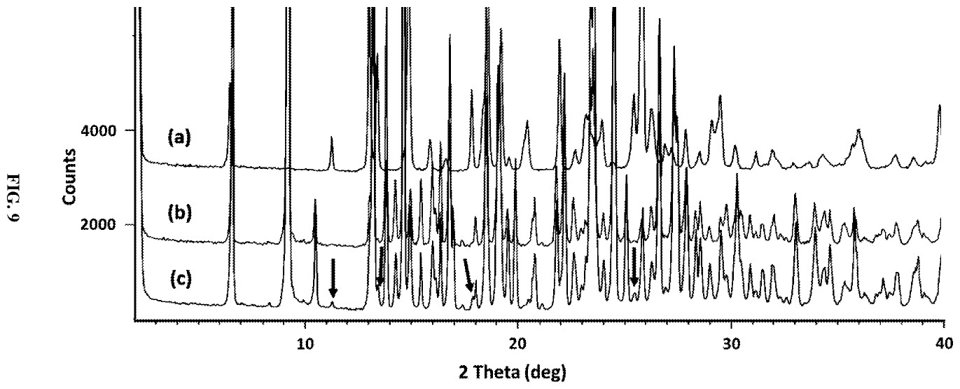

46.3 g of meloxicam and 28.48 g of acetylsalicylic acid were combined and then 231.5 mL of pure ethyl acetate (1:5 w/v) was then added to the mixture. The reaction mass was stirred for 16 hours at 200 rpm. 92.6 mL of additional pure ethyl acetate was added and the mixture was stirred for an additional 3 hrs. The resultant slurry was then filtered and solids washed with pure ethyl acetate. The solid crystalline material was dried in an oven at 40° C. for 24 hours. The particulate material was gathered and stored in screw cap containers for subsequent analysis. A comparison of the improved purity of the co-crystal as compared to the prior art process of U.S. Pat. No. 8,124,603 is illustrated in FIG. 9. Meloxicam contamination can be seen by the presence of characteristic peaks at 11.3, 13.4, 17.9, 25.4) (° 2 theta as indicated by the arrows.

### Example 9. Process for the Preparation of Co-Crystal Meloxicam:Salicylic Acid Form III (1:1) Via Process 2

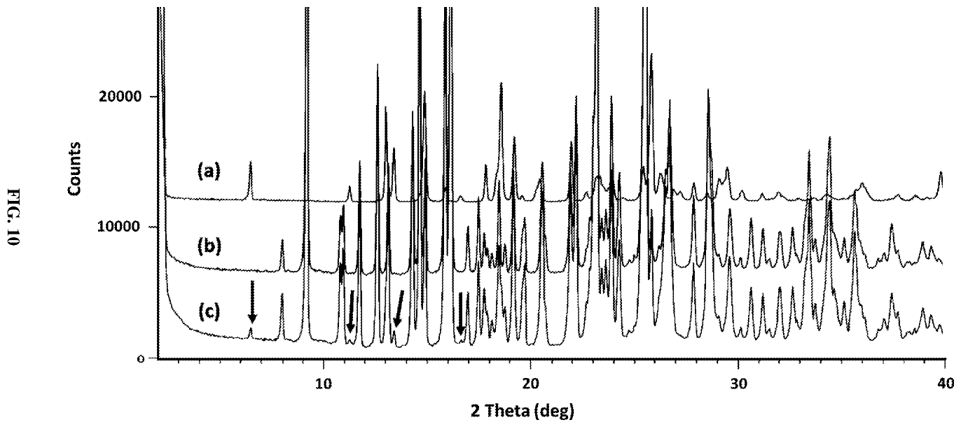

50 g of meloxicam and 25.55 g of salicylic acid were combined and then 250 mL of pure ethyl acetate (1:5 w/v) was then added to the mixture. The reaction mass was stirred for 16 hours at 200 rpm. 100 mL of additional pure ethyl acetate was added and the mixture was stirred for an additional 3 hrs. The resultant slurry was filtered and solids washed with pure ethyl acetate. The solid crystalline material was dried in an oven at 40° C. for 24 hours. The particulate material was gathered and stored in screw cap containers for subsequent analysis. A comparison of the improved purity of the co-crystal as compared to the prior art process of U.S. Pat. No. 8,124,603 is illustrated in FIG. 10. Meloxicam contamination can be seen by the presence of characteristic peaks at 6.5, 11.3, 13.4, 16.6(°) 2 theta as indicated by the arrows.

### Example 10. Process for the Preparation of Co-Crystal Meloxicam:Maleic Acid (1:1) Via Process 2

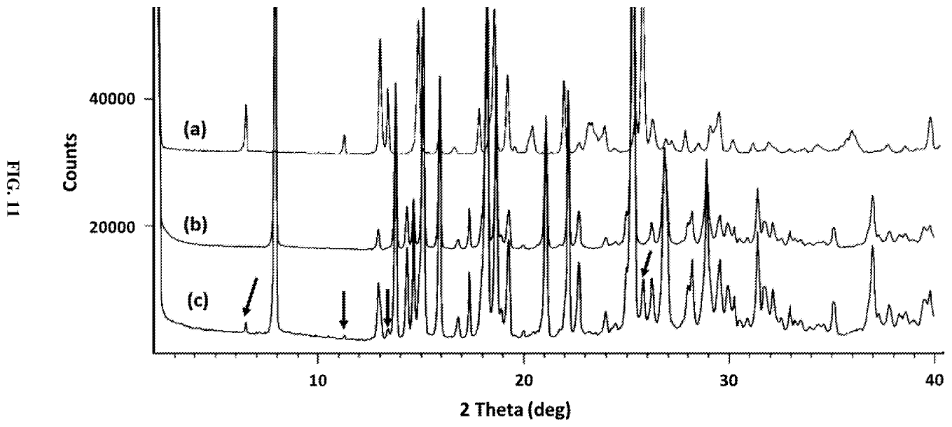

20 g of meloxicam and 8.58 g of maleic acid were combined and then 100 mL of pure ethyl acetate (1:5 w/v) was then added to the mixture. The reaction mass was stirred for 16 hours at 200 rpm. 40 mL of additional pure ethyl acetate was added and the mixture was stirred for an additional 3 hrs. The resultant slurry was filtered and solids washed with pure ethyl acetate. The solid crystalline material was dried in an oven at 40° C. for 24 hours. The particulate material was gathered and stored in screw cap containers for subsequent analysis. A comparison of the improved purity of the co-crystal as compared to the prior art process of U.S. Pat. No. 8,124,603 is illustrated in FIG. 11. Meloxicam contamination can be seen by the presence of characteristic peaks at 6.5, 11.3, 13.4, 25.8(°) 2 theta as indicated by the arrows.

### Example 11. Process for the Preparation of Co-Crystal Meloxicam:Succinic Acid (2:1) Via Process 2

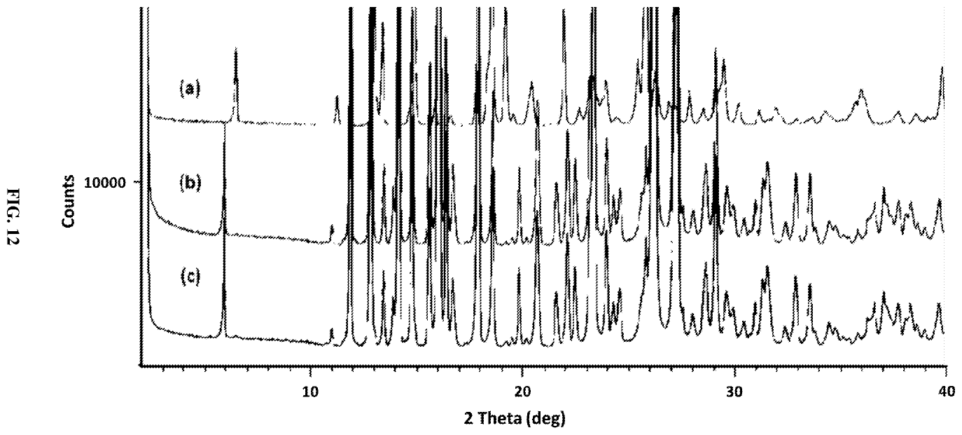

Meloxicam (50 g) and ethyl acetate (250 mL, 1:5 w/v) were combined and the suspension was stirred for 30-60 min. Succinic acid (9.24 g, 0.55 mol) was added to the suspension, and the contents were stirred at 30±5° C. for 15-16 hours. The slurry was filtered, and the cake washed with ethyl acetate (0.6 vol, twice). The solids were vacuum dried for 30-60 min. followed by drying at 45±5° C. under vacuum for 15-16 hours. The material was then cooled to 30±5° C. and milled. The milled material was dried at 45±5° C. under vacuum for 6-8 hours, until the ethyl acetate content is NMT 5000 ppm. The obtained solid was checked by PXRD and identified as co-crystal of meloxicam:succinic acid (2:1). PXRD comparison is shown in FIG. 12.

### Example 12. Process for the Preparation of Co-Crystal Meloxicam:Succinic Acid (2:1) with Higher Mole Ratios of Succinic Acid Via Process 2

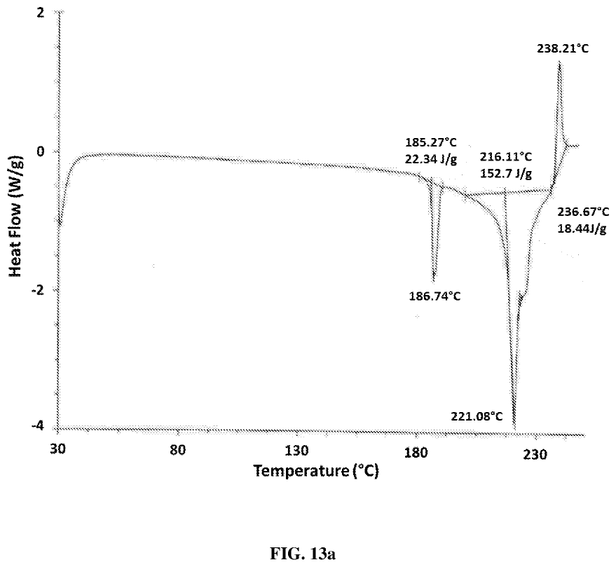

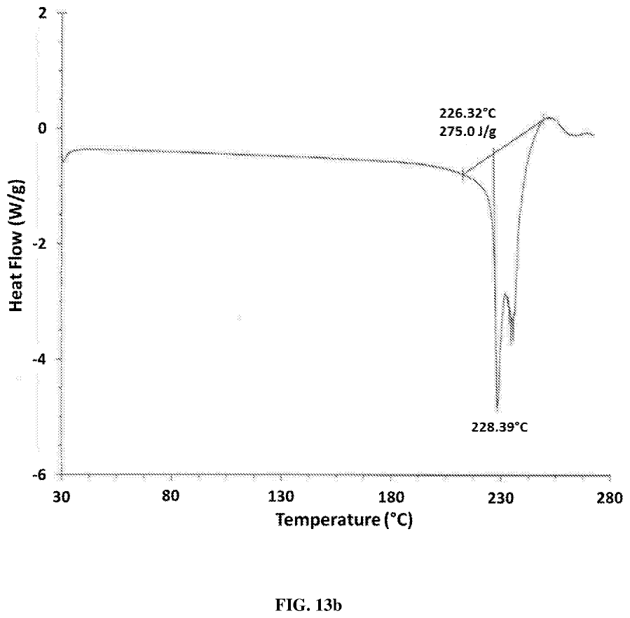

Meloxicam (20 g) and ethyl acetate (160 mL, 1:8 w/v) were combined and the suspension was stirred for 30-60 min. Succinic acid (6.72 g, 1.0 mol) was added to the suspension, and the contents were stirred at 30±5° C. for 15-16 hours. The slurry was filtered, and the cake washed with ethyl acetate (0.6 vol, twice). The solids were vacuum dried for 30-60 min. followed by drying at 45±5° C. under vacuum for 24 hours. The obtained solid was checked by PXRD which is similar to co-crystal of meloxicam:succinic acid (2:1) (see FIG. 12). However, the DSC thermogram (FIG. 13a) of the solid shows a small endothermic peak at about 186.74° C. attributing to presence of trace amounts of succinic acid. The product (25 g) thus obtained was then suspended in water (250 mL), heated to 50±5° C. and maintained under stirring for 2 hours. The reaction mass was then filtered at 50±5° C. and dried at 45±5° C. for 15 hours. The obtained solid was checked by PXRD (similar to FIG. 12) and DSC (FIG. 13b) and identified as co-crystal of meloxicam:succinic acid (2:1). The DSC data after water leaching shows a single melting endotherm at about 228.39° C. with complete absence of succinic acid contamination.

### Example 13. Preparation of Co-Crystal of Meloxicam:Acetylsalicylic Acid (1:1) Via Process 2

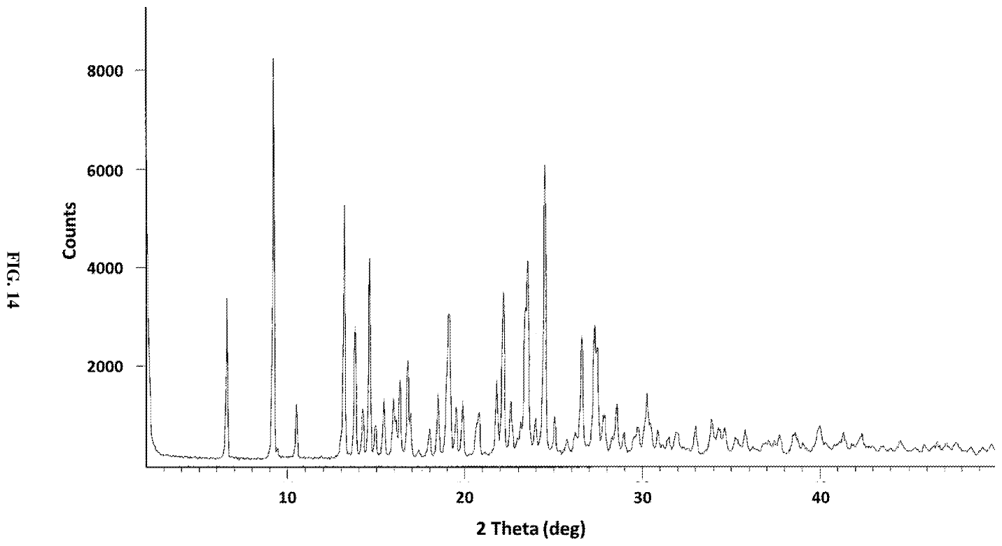

100 g of meloxicam, 71.5 g (1.4 mole eq.) of acetylsalicylic acid were charged into the round bottom flask. Added 500 mL of particle free ethyl acetate (1:5 w/v) at 25±5° C. Stirred the contents for 10-15 min at 25±5° C. The reaction mass was then cooled to 6±3° C., stirred at this temperature for 16 hours. The resultant slurry was then filtered, and wet cake washed with pre-cooled ethyl acetate at 6±3° C. and suck dried for 30 minutes. Solid material obtained was dried under vacuum at 28±5° C. for 120 minutes and further dried under vacuum at 45±2° C. for 8 hours. The product obtained was checked by PXRD (FIG. 14) and identified as co-crystal of meloxicam:acetylsalicylic acid (1:1).

### Example 14. Process for the Preparation of Co-Crystal of Meloxicam:Salicylic Acid Form III (1:1) Via Process 2

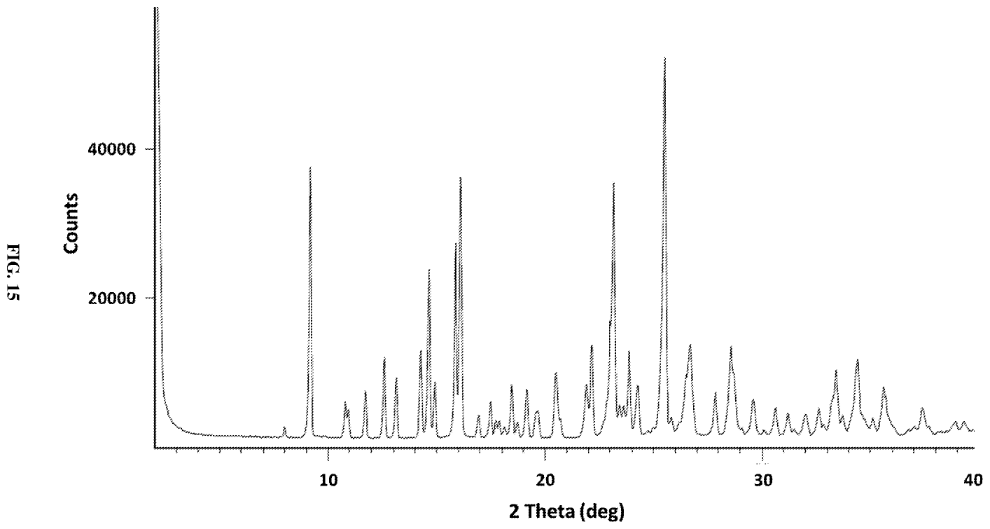

100 g of meloxicam, 58.95 g (1.50 mole eq.) of salicylic acid were charged into the round bottom flask. Added 500 mL of particle free ethyl acetate (1:5 w/v) at 25±5° C. The reaction mass was stirred for 16 hours at 24±3° C. The resultant slurry was filtered, and wet cake was washed with ethyl acetate 100 mL. The wet solid was suck dried for 30 minutes. The solid material was vacuum dried for 120 minutes at 30±5° C. and further dried under vacuum at 50±2° C. for 8 hours. The product obtained was checked by PXRD (FIG. 15) and identified as co-crystal of meloxicam:salicylic acid Form III (1:1).

### Example 15. Process for the Preparation of Co-Crystal of Meloxicam:Maleic Acid (1:1) Via Process 2

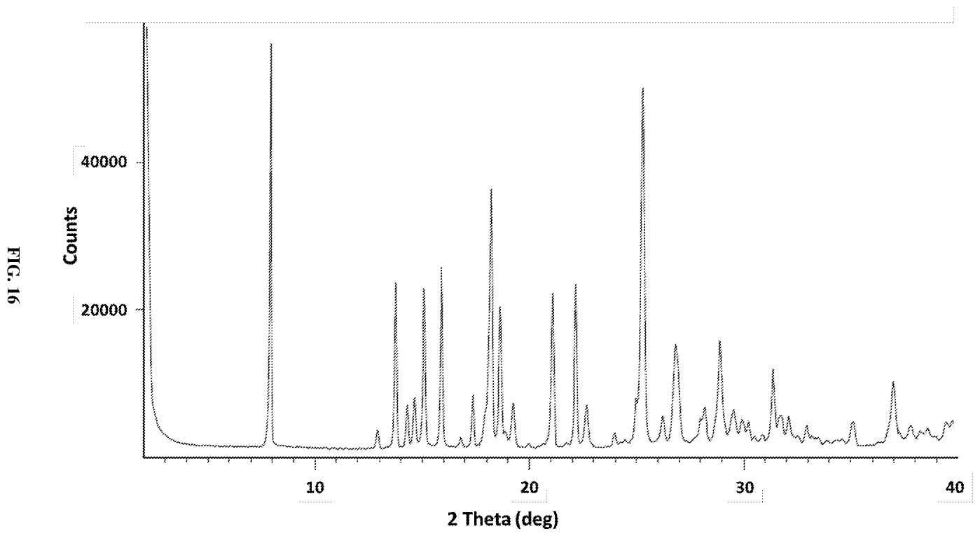

100 g of meloxicam and 42.94 g (1.30 mole. eq.) of maleic acid were charged into the round bottom flask. Added 500 mL of particle free ethyl acetate (1:5 w/v) at 25±5° C. The reaction mass was stirred for 16 hours at 24±3° C. Ethyl acetate 200 mL was added and the reaction mass was stirred for an additional 3 hours at 24±3° C. The resultant slurry was filtered, wet cake was washed with ethyl acetate (100 mL) and suck dried for 120 minutes. The solid material obtained was vacuum dried for 120-240 minutes at 30±5° C. and further dried under vacuum at 50±2° C. for 12 hours. The product obtained was checked by PXRD (FIG. 16) and identified as co-crystal of meloxicam:maleic acid (1:1).

### Example 16. Process for the Preparation of Co-Crystal of Meloxicam:Succinic Acid (2:1) Via Process 2

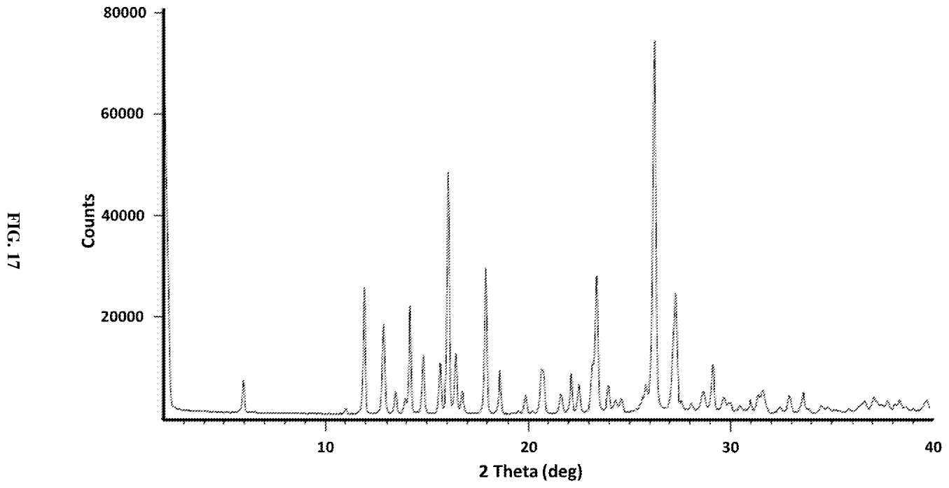

Charged 4.5 kg of meloxicam and ethyl acetate (18.0 L) into reactor at 30±5° C. Stirred the contents for 30-60 minutes at 30±5° C. Charged succinic acid (0.831 kg, 0.55 mole eq.) and ethyl acetate (4.5 L, 1:5 w/v, total) into the reactor at 30±5° C. The suspension was stirred at 30±5° C. for 15-16 hours at 110 RPM. The resulting slurry was filtered, and the cake was washed with ethyl acetate (2.7 L, twice). The solids were vacuum dried for 30-60 minutes at 30±5° C. followed by drying at 45±5° C. under vacuum for 15-16 hours. The material was then cooled to 30±5° C. and milled. The milled material was dried at 45±5° C. under vacuum for 8 hours, until the ethyl acetate content is NMT 5000 ppm. The product obtained was checked by PXRD (FIG. 17) and identified as co-crystal of meloxicam:succinic acid (2:1).

### Example 17. Process for the Preparation of Co-Crystal of Meloxicam:Succinic Acid (2:1) Via Process 2

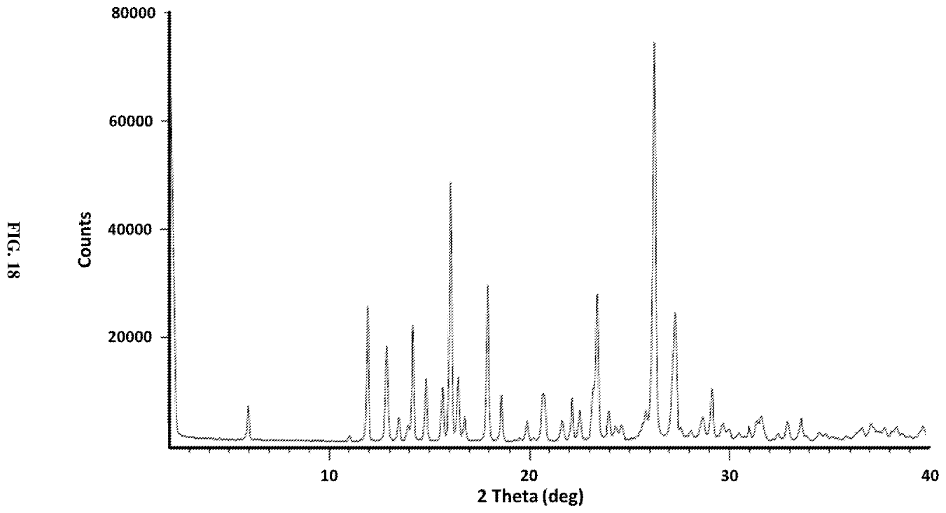

100 g of meloxicam and 18.48 g (0.55 mole eq.) of succinic acid were charged into the round bottom flask. Added 500 mL of ethyl acetate (1:5 w/v) at 30±5° C. The suspension was stirred at 30±5° C. for 15-16 hours. The slurry was filtered, and the cake washed with ethyl acetate (60 mL, twice). The solids were vacuum dried for 30-60 minutes followed by drying at 47±3° C. under vacuum for 15-16 hours. The material was then cooled to 30±5° C. and milled. The milled material was dried at 47±3° C. under vacuum for 8 hours, until the ethyl acetate content is NMT 5000 ppm. The product obtained was checked by PXRD (FIG. 18) and identified as co-crystal of meloxicam:succinic acid (2:1).

### Example 18. Solid State Stability

The physical and chemical stability of co-crystal of meloxicam:acetylsalicylic acid (1:1), co-crystal of meloxicam:1-hydroxy-2-naphthoic acid and co-crystal of meloxicam:salicylic acid Form III, obtained as per Examples 1, 2, and 3, respectively, was tested by storing the samples at 40° C./75% relative humidity (RH) and at 25° C./60% relative humidity (RH) for three months. The samples were analyzed for PXRD and assayed by HPLC according to the methods described above; results are shown Table 3. Each co-crystal was found to be physically and chemically stable at 40° C./75% relative humidity (RH) and at 25° C./60% relative humidity (RH) up to three months.

TABLE 3

| | Meloxicam:Acetylsalicylic | | | Meloxicam:1-Hydroxy-2- | | | Meloxicam:Salicylic acid | | |
|---|---|---|---|---|---|---|---|---|---|
| | acid (1:1) | | | Naphthoic acid (1:1) | | | Form III (1:1) | | |
| Time | PXRD | M % | A % | PXRD | M % | A % | PXRD | M % | A % |

at 40° C./75% RH

| | | | | | | | | | |
|---|---|---|---|---|---|---|---|---|---|
| Initial | X | 65.7 | 33.6 | X | 64.3 | 34.2 | X | 71.3 | 27.5 |
| 1 mo. | S | 65.9 | 33.4 | S | 64.9 | 34.9 | S | 72.0 | 27.8 |
| 2 mo. | S | 65.8 | 32.8 | S | 64.8 | 34.8 | S | 71.5 | 27.0 |
| 3 mo. | S | 65.4 | 33.0 | S | 65.5 | 34.2 | S | 71.8 | 27.4 |

at 25° C./60% RH

| | | | | | | | | | |
|---|---|---|---|---|---|---|---|---|---|
| Initial | X | 65.7 | 33.6 | X | 64.3 | 34.2 | X | 71.3 | 27.5 |
| 1 mo. | S | 66.0 | 33.4 | S | 64.7 | 34.5 | S | 71.7 | 27.8 |
| 2 mo. | S | 66.0 | 33.0 | S | 65.0 | 35.0 | S | 72.0 | 27.2 |
| 3 mo. | S | 65.7 | 33.0 | S | 64.6 | 34.0 | S | 72.1 | 27.6 |

X: crystalline; S: stable.
M %: Meloxicam Assay %; A %: Co-former Assay %.

### Example 15. Purity Assay

Each co-crystal of meloxicam:1-hydroxy-2-naphthoic acid (1:1) was assayed by HPLC and GC-HS according to the methods described above; results are shown Table 4. The analytical results for co-crystal of meloxicam:acetylsalicylic acid (1:1), co-crystal of meloxicam:salicylic acid Form III (1:1), co-crystal of meloxicam:maleic acid (1:1) and co-crystal of meloxicam:succinic acid (2:1) obtained as per Examples 13, 14, 15, 16, and 17, respectively, are given in Table 4a.

TABLE 4
Co-crystal of Meloxicam:1-Hydroxy-2-Naphthoic acid (1:1)

| | Impurities | | | | | | |
|---|---|---|---|---|---|---|---|
| | (% w/w, by HPLC)^{#} | | | | Assay | | Residual solvent |
| | Methyl | Ethyl | | | (% by HPLC) | | (ppm, by GC-HS) |
| Ex. | Meloxicam | Meloxicam | Other* | Total | M % | A % | DMSO |
| 6 | ND | ND | 0.03 | 0.09 | 65.18 | 35.99 | 584 |
| 7 | 0.01 | ND | 0.02 | 0.05 | 65.1 | 35.0 | 365 |

M %: Meloxicam Assay %; A %: Co-former Assay %.
ND: not detected.
*Any other individual impurity.
^{#}Meloxicam Related Compounds A, B, C, and D (per the Meloxicam USP Monograph, USP35-3789) were not detectable at any timepoint from initiation to 3 months. USP Meloxicam Related Compound A: Ethyl 4-hydroxy-2-methyl-2H-1,2-benzothiazine-3-carboxylate 1,1-dioxide; USP Meloxicam Related Compound B: 5-Methylthiazol-2-amine; USP Meloxicam Related Compound C: Isopropyl-4-hydroxy-2-methyl-2H-1,2-benzothiazine-3-carboxylate-1,1-dioxide; and USP Meloxicam Related Compound D: 4-Methoxy-2-methyl-N-(5-methyl-1,3-thiazole-2-yl)-2H-1,2-benzothiazine-3-carboxamide-1,1-dioxide.

TABLE 4a

| | Impurities | | | | | | |
|---|---|---|---|---|---|---|---|
| | (% w/w, by HPLC)^{#} | | | | Assay | | Residual solvent |
| | Methyl | Ethyl | | | (% by HPLC) | | (ppm, by GC-HS) |
| Ex. | Meloxicam | Meloxicam | Other* | Total | M % | A % | Ethyl Acetate |
| 13 | ND | ND | 0.01 | 0.02\hat{ } | 66.2 | 32.8 | 3183 |
| 14 | 0.00 | ND | 0.01 | 0.01\hat{ }\hat{ } | 71.1 | 27.8 | 2042 |
| 15 | ND | ND | 0.01 | 0.01 | 74.9 | 25.0 | 860 |
| 16 | ND | ND | 0.01 | 0.03 | 86.09 | 14.26 | 1815 |
| 17 | 0.01 | ND | 0.03 | 0.06 | 85.1 | 14.2 | 3008 |

M %: Meloxicam Assay %; A %: Co-former Assay %.
ND: not detected.
\hat{ }includes 0.015% salicylic acid; \hat{ }\hat{ }salicylic acid not detected.
*Any other individual impurity.
^{#}Meloxicam Related Compounds A, B, C, and D (per the Meloxicam USP Monograph, USP35-3789) were not detectable at any timepoint from initiation to 3 months. USP Meloxicam Related Compound A: Ethyl 4-hydroxy-2-methyl-2H-1,2-benzothiazine-3-carboxylate 1,1-dioxide; USP Meloxicam Related Compound B: 5-Methylthiazol-2-amine; USP Meloxicam Related Compound C: lsopropyl-4-hydroxy-2-methyl-2H-1,2-benzothiazine-3-carboxylate-1,1-dioxide; and USP Meloxicam Related Compound D: 4-Methoxy-2-methyl-N-(5-methyl-1,3-thiazole-2-yl)-2H-1,2-benzothiazine-3-carboxamide-1,1-dioxide.

### Example 19. Solid State Stability & Purity

The physical and chemical stability of meloxicam:1-hydroxy-2-naphthoic acid (1:1) obtained as per Example 7 was tested by storing the samples at 40° C./75% relative humidity (RH) and at 25° C./60% relative humidity (RH) for three months. The samples were analyzed for PXRD and assayed by HPLC according to the methods described above; results are shown Table 5. The co-crystal was found to be physically and chemically stable at 40° C./75% relative humidity (RH) and at 25° C./60% relative humidity (RH) at least three months.

TABLE 5
Co-crystal of Meloxicam:1-Hydroxy-2-Naphthoic acid (1:1)

| | Impurities | | | | | | |
|---|---|---|---|---|---|---|---|
| | (% w/w, by HPLC)^{#} | | | | Assay | | |
| | Methyl | Ethyl | | | (% by HPLC) | | |
| Time | Meloxicam | Meloxicam | Other* | Total | M % | A % | PXRD |

at 40° C./75% RH

| | | | | | | | |
|---|---|---|---|---|---|---|---|
| Initial | 0.01 | ND | 0.03 | 0.06 | 65.2 | 35.0 | X |
| 1 mo. | 0.01 | ND | 0.03 | 0.09 | 64.7 | 35.0 | S |
| 2 mo. | 0.01 | ND | 0.03 | 0.13 | 64.0 | 35.2 | S |
| 3 mo. | 0.01 | ND | 0.03 | 0.10 | 64.4 | 35.4 | S |

at 25° C./60% RH

| | | | | | | | |
|---|---|---|---|---|---|---|---|
| Initial | 0.01 | ND | 0.02 | 0.05 | 65.1 | 35.0 | X |
| 1 mo. | 0.01 | ND | 0.03 | 0.08 | 65.0 | 35.1 | S |
| 2 mo. | 0.01 | ND | 0.03 | 0.13 | 63.5 | 34.8 | S |
| 3 mo. | 0.01 | ND | 0.03 | 0.08 | 64.3 | 35.2 | S |

M %: Meloxicam Assay %; A %: Co-former Assay %. ND: not detected. X: crystalline; S: stable.
*Any other individual impurity.
^{#}Meloxicam Related Compounds A, B, C, and D (see Table 4) were not detectable at any timepoint from initiation up to 3 months.

The physical and chemical stability of co-crystal of meloxicam:succinic acid (2:1) obtained as per Example 17 was tested by storing the samples at 40° C./75% relative humidity (RH) and at 25° C./60% relative humidity (RH) for three months as mentioned in below Table 5a. The samples were analyzed by PXRD, assay and related substances by HPLC. The co-crystal of meloxicam:succinic acid (2:1) was found to be physically and chemically stable at 40° C./75% relative humidity (RH) and at 25° C./60% relative humidity (RH) at least three months.

TABLE 5a
Co-crystal of Meloxicam:Succinic acid (2:1)

| | Impurities | | | | | | |
|---|---|---|---|---|---|---|---|
| | (% w/w, by HPLC)^{#} | | | | Assay | | |
| | Methyl | Ethyl | | | (% by HPLC) | | |
| Time | Meloxicam | Meloxicam | Other* | Total | M % | A % | PXRD |

at 40° C./75% RH

| | | | | | | | |
|---|---|---|---|---|---|---|---|
| Initial | 0.01 | BDL | 0.03 | 0.06 | 85.1 | 14.2 | X |
| 1 mo. | 0.01 | BDL | 0.03 | 0.14 | 84.8 | 14.1 | S |
| 2 mo. | BDL | BDL | 0.03 | 0.12 | 83.9 | 14.3 | S |
| 3 mo. | BDL | BDL | 0.01 | 0.08 | 84.9 | 14.1 | S |

at 25° C./60% RH

| | | | | | | | |
|---|---|---|---|---|---|---|---|
| Initial | 0.01 | BDL | 0.03 | 0.06 | 85.1 | 14.2 | X |
| 1 mo. | 0.01 | BDL | 0.03 | 0.14 | 85.2 | 13.9 | S |
| 2 mo. | BDL | BDL | 0.03 | 0.13 | 85.1 | 14.2 | S |
| 3 mo. | BDL | BDL | 0.02 | 0.13 | 84.8 | 14.1 | S |

M %: Meloxicam Assay %; A %: Co-former Assay %. ND: not detected. X: crystalline; S: stable.
*Any other individual impurity.
^{#}Meloxicam Related Compounds A, B, C, and D (see Table 4a) were not detectable at any timepoint from initiation up to 3 months.

The physical and chemical stability of co-crystal of meloxicam:salicylic acid Form III (1:1) obtained as per Example 14 was tested by storing the samples at 40° C./75% relative humidity (RH) for two months as mentioned in below Table 5b. The samples were analyzed by PXRD, assay and related substances by HPLC. The co-crystal of meloxicam:salicylic acid Form III (1:1) was found to be physically and chemically stable at 40° C./75% relative humidity (RH) at least 2 months.

TABLE 5b
Co-crystal of Meloxicam:Salicylic acid Form III (1:1)

| | Impurities | | | | | | |
|---|---|---|---|---|---|---|---|
| | (% w/w, by HPLC)^{#} | | | | Assay | | |
| | Methyl | Ethyl | | | (% by HPLC) | | |
| Time | Meloxicam | Meloxicam | Other* | Total | M % | A % | PXRD |

at 40° C./75% RH

| | | | | | | | |
|---|---|---|---|---|---|---|---|
| Initial | ND | ND | 0.01 | 0.01 | 71.1 | 27.8 | X |
| 1 mo. | ND | 0.003 | 0.01 | 0.04 | 71.4 | 28.4 | S |
| 2 mo. | 0.001 | ND | 0.01 | 0.01 | 71.2 | 28.2 | S |

M %: Meloxicam Assay %; A %: Co-former Assay %. ND: not detected. X: crystalline; S: stable.
*Any other individual impurity.
^{#}Meloxicam Related Compounds A, B, C, and D (see Table 4a) were not detectable at any timepoint from initiation up to 2 months.

### Example 20. Contamination of Meloxicam/API

The residual contamination of meloxicam API (“free meloxicam”) was tested in co-crystals prepared by the preceding Examples through PXRD analysis with PXRD scan times of 30 minutes according to the “X-ray powder diffraction analysis of meloxicam co-crystals (PXRD)” method described herein. The detection limit for free meloxicam in the co-crystals in the preceding PXRD method were found to be not more than 1 wt. % or not more than 0.3 wt. % with increasing scan time to 30 minutes. Improved purity of the co-crystals is observed as compared to the prior art process of U.S. Pat. No. 8,124,603 based on the studies conducted with the same extended PXRD method (Table 6).

TABLE 6

| | |
|---|---|
| | Meloxicam/API contamination |
| Reference examples | by PXRD (wt. %) |

As per U.S. Pat. No. 8,124,603

| | |
|---|---|
| Example 43, Meloxicam:Acetylsalicylic acid (1:1) | >1% |
| Example 44, Meloxicam:Salicylic Acid Form III (1:1) | >1% |
| Example 45, Meloxicam:1-Hydroxy-2-Naphthoic acid (1:1) | >1% |
| Example 46, Meloxicam:Succinic Acid (2:1) | BDL |

This Application

| | |
|---|---|
| Prepared by Co-Crystallization Methods | |
| Example 1, Meloxicam:Acetylsalicylic acid (1:1) | BDL |
| Example 2, Meloxicam:1-Hydroxy-2-Naphthoic acid (1:1) | BDL |
| Example 3, Meloxicam:Salicylic Acid Form III (1:1) | BDL |
| Example 6, Meloxicam:1-Hydroxy-2-Naphthoic acid (1:1) | BDL |
| Example 7, Meloxicam:1-Hydroxy-2-Naphthoic acid (1:1) | BDL |
| Prepared by Slurry Methods | |
| Example 13, Meloxicam:Acetylsalicylic acid (1:1) | BDL |
| Example 14, Meloxicam:Salicylic acid Form III (1:1) | BDL |
| Example 15, Meloxicam:Maleic acid (1:1) | BDL |
| Example 16, Meloxicam:Succinic acid (2:1) | BDL |
| Example 17, Meloxicam:Succinic acid (2:1) | BDL |

*BDL: Indicates below detection limit

It is to be understood that the description of the present disclosure has been simplified to illustrate elements that are relevant for a clear understanding of the invention, while eliminating, for purposes of clarity, other elements that may be well known. In view of the above description, accompanying drawing figures, and the examples, one of ordinary skill in the art will be able to practice the instant description without undue experimentation. The foregoing will be better understood with reference to the following examples that detail certain procedures for the preparation of molecules and compositions described herein. All references made to the examples should not be considered exhaustive, nor limiting, but merely illustrative of only a few of the many aspects and embodiments contemplated by the present disclosure.

1. A substantially pure co-crystal of meloxicam with a co-former selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, succinic acid, maleic acid, salicylic acid, fumaric acid, adipic acid, DL-malic acid, L-malic acid, glutaric acid, malonic acid, glycolic acid, camphoric acid, maltol, and ethyl maltol.

2. The co-crystal of claim 1, wherein the co-former is 1-hydroxy-2-naphthoic acid, acetylsalicylic acid, salicylic acid, succinic acid, or maleic acid.

3. The co-crystal of claim 1, having less than about 2 wt. % total impurities.

4. (canceled)

5. The co-crystal of claim 1, having an absence of one or more PXRD signals attributable to free meloxicam selected from the group consisting of 6.5, 11.2, 13.2, 14.9, and 17.8°+/−0.2° 2Θ.

6. The co-crystal of claim 5, wherein the co-former is 1-hydroxy-2-naphthoic acid, and the co-crystal has an absence of the PXRD signal attributable to free meloxicam at 13.2°+/−0.2° 2Θ.

7. The co-crystal of claim 5, wherein the co-former is acetylsalicylic acid, and the co-crystal has an absence of the PXRD signal attributable to free meloxicam at 11.2°+/−0.2° 2Θ.

8. The co-crystal of claim 5, wherein the co-former is salicylic acid, and the co-crystal has an absence of the PXRD signal attributable to free meloxicam at 6.5°+/−0.2Θ 2Θ.

9. The co-crystal of claim 5, wherein the co-former is succinic acid, and the co-crystal has an absence of the PXRD signal attributable to free meloxicam at 13.2°+/−0.2Θ 2Θ.

10. The co-crystal of claim 5, the co-former is maleic acid, and the co-crystal has an absence of the PXRD signal attributable to free meloxicam at 6.5°+/−0.2Θ 2Θ.

11. The co-crystal of claim 1, having less than 1 wt. % total impurities as measured by HPLC after storing a sample of the co-crystal at 40+/−5° C. and 75+/−5% relative humidity (RH) or at 25° C. and 60% RH for three months.

12. (canceled)

13. A process for preparing a meloxicam co-crystal comprising:
combining a solution comprising meloxicam, a co-former, and an organic solvent, with an anti-solvent; and
isolating the meloxicam co-crystal.

14. The process of claim 13, wherein the co-former is selected from the group consisting of 1-hydroxy-2-naphthoic acid, acetylsalicylic acid, benzoic acid, 2,5-dihydroxybenzoic acid, 4-hydroxybenzoic acid, hydrocinnamic acid, and salicylic acid.

15. The process of claim 13, wherein the organic solvent comprises a polar aprotic solvent.

16. The process of claim 15, wherein the organic solvent comprises dimethyl sulfoxide, N,N-dimethylformamide, N,N-dimethylacetamide, N-methylpyrrolidinone, formamide, nitromethane, acetonitrile, dimethyl carbonate, diethyl carbonate, ethylene carbonate, propylene carbonate, or a mixture thereof.

17. The process of claim 15, wherein the organic solvent comprises dimethyl sulfoxide.

18. The process of claim 13, further comprising forming the solution by heating the organic solvent, meloxicam, and co-former.

19. (canceled)

20. A process for preparing a meloxicam co-crystal comprising:
forming a suspension of meloxicam and a co-former in an organic solvent,
agitating the suspension for a period of time suitable to provide a meloxicam co-crystal, and
isolating the meloxicam co-crystal; wherein
either the ratio of meloxicam measured in grams to organic solvent measured in mL (“the w/v ratio”) in the suspension is greater than about 1:5; or
one of the meloxicam or the co-former is present in the suspension in at least a 10% molar excess with respect to the other.

21. The process of claim 20, wherein the w/v ratio is greater than about 1:5; and one of the meloxicam or the co-former is present in the suspension in at least a 10% molar excess with respect to the other.

22. The process of claim 20, wherein the co-former is selected from the group consisting of 1-hydroxy-2-naphthoic acid, acetylsalicylic acid, benzoic acid, 2,5-dihydroxy-benzoic acid, 4-hydroxybenzoic acid, hydrocinnamic acid, succinic acid, maleic acid, salicylic acid, fumaric acid, adipic acid, DL-malic acid, L-malic acid, glutaric acid, malonic acid, glycolic acid, camphoric acid, maltol, and ethyl maltol.

23.-25. (canceled)

26. The process of claim 20, further comprising contacting the meloxicam co-crystal with water for a suitable period of time to remove at least a portion of a water-soluble impurity.
